SEQUENCE LISTING (11 sequences)

SEQ ID NO: 1 - protein, 120 aa, Artificial Sequence
  Synthetic Construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVS

SEQ ID NO: 2 - protein, 109 aa, Artificial Sequence
  Synthetic Construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRT

SEQ ID NO: 3 - protein, 10 aa, Artificial Sequence
  Synthetic Construct
GFSLIGYDLN

SEQ ID NO: 4 - protein, 16 aa, Artificial Sequence
  Synthetic Construct
IIWGDGTTDYNSAVKS

SEQ ID NO: 5 - protein, 13 aa, Artificial Sequence
  Synthetic Construct
GGYWYATSYYFDY

SEQ ID NO: 6 - protein, 11 aa, Artificial Sequence
  Synthetic Construct
RASQSISNNLN

SEQ ID NO: 7 - protein, 7 aa, Artificial Sequence
  Synthetic Construct
YTSRFHS

SEQ ID NO: 8 - protein, 9 aa, Artificial Sequence
  Synthetic Construct
QQEHTLPYT

SEQ ID NO: 9 - protein, 447 aa, Artificial Sequence
  Synthetic Construct
  MISC_FEATURE (447)..(447): C-terminal lysine (K) at position 447 is optional
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Artificial Sequence
  Synthetic Construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, Artificial Sequence
  Synthetic Construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

(19) United States
(12) Patent Application Publication
(10) Pub. No.: US 2022/0135663 A1
(43) Pub. Date: May 5, 2022

(54) Method of treatment of Chronic Low Back Pain

(71) Applicant: Pfizer Inc., New York, NY (US)

(72) Inventors: Christine Ruth WEST, Groton, CT (US); Mark Thomas BROWN, Groton, CT (US); Candace Roskoph BRAMSON, Groton, CT (US)

(21) Appl. No.: 17/430,079

(22) PCT Filed: Feb. 14, 2020
(86) PCT No.: PCT/IB2020/051281

§ 371 Date: Aug. 11, 2021

Related U.S. Application Data
(60) Provisional application No. 62/963,678, filed on Jan. 21, 2020.
(60) Provisional application No. 62/879,615, filed on Jul. 29, 2019.
(60) Provisional application No. 62/811,701, filed on Feb. 28, 2019.
(60) Provisional application No. 62/807,025, filed on Feb. 18, 2019.

(51) Int. Cl. C07K 16/22; A61P 29/00

(52) U.S. Cl. CPC C07K 16/22; A61P 29/00

(57) ABSTRACT

The present invention relates to the treatment of chronic low back pain with an anti-nerve growth factor (NGF) antibody.

### FIELD

The present invention relates to the treatment of chronic low back pain with an anti-nerve growth factor (NGF) antibody.

### BACKGROUND

Chronic low back pain, generally defined as back pain that persists more than 12 weeks, represents a significant cause of morbidity, disability, and lost productivity world-wide (Borenstein D. Musculoskeletal Medicine 1996; 22 (3); 439-456). Estimates of the prevalence of chronic low back pain vary by study and by geographic region, but chronic low back pain is a common cause of chronic pain and disability in all regions studied. In the United States, the prevalence of chronic low back pain derived from the 1988 United States National Health Interview Survey was 6.4% (Praemer et al., Rosemont: AAOS, 1992: 1-99) and a survey in 2008 estimated the prevalence of chronic low back pain to be 8.1% (Johannes et al., Journal of Pain; 2010; 11 (11): 1230-1239). Estimates in Europe suggest that the prevalence of non-specific chronic low back pain is 23%; with 11-12% of the population being disabled by low back pain (Airakinsinen et al., Eur Spine J; 2006; 15 (Suppl. 2): S192-S300). In Japan, a large population-based survey found that 3.87% of the population had experienced chronic, disabling low back pain during their lifetime (Tomoko et al., Eur Spine J. Published online: 7 Aug. 2012).

The majority of chronic low back pain cannot be attributed to a single pathophysiological or anatomical cause but is usually multifactorial in nature. This back pain is often called “mechanical” or “non-specific” low back pain (Deyo et al., N Engl J Med 2001; 344: 363-370). Back pain may originate from many spinal structures including facet joints, ligaments, paravertebral musculature, intervertebral discs, and nerves. Common causes of low back pain include injuries to the musculoligamentous structures, age-related degenerative processes of the discs and facet joints, spinal stenosis, and disc herniation (Deyo R A, et al., N Engl J Med 2001; 344: 363-370). Given the lack of a specific etiology in most cases, therapeutic measures are aimed at providing symptomatic relief and restoring function.

Pharmacological agents commonly used to treat chronic low back pain include nonsteroidal anti-inflammatory drugs (NSAIDs), tricyclic antidepressants, muscle relaxants, opioid analgesics, and other drugs active in the central nervous system. However, these agents are not fully effective in many patients, and the use of these agents can be limited by side effects such as gastrointestinal bleeding, somnolence and cognitive impairment. There are, in addition, a variety of other care modalities, such as epidural injections, nerve blocks, facet joint injections, implanted electrical stimulators and pumps, physical therapy, chiropractic and acupuncture, which are expensive and unproven and still leave many patients with inadequate pain relief. Pharmacologic management of pain not responsive to NSAIDs without the toxicities of opiates is needed for patients experiencing moderate-to-severe chronic low back pain.

Development of novel pharmacologic therapies targeting the function of key pain modulators may provide new treatment options with improved efficacy and/or safety. Nerve growth factor (NGF) is a neurotrophin and key mediator of pain, with a demonstrated role in pain signal transduction and pathophysiology. Tanezumab is a humanized anti-NGF monoclonal antibody that has high specificity and affinity for NGF, thereby blocking binding of NGF to its receptors, TrkA and p75 (Abdiche et al. Protein Sci. 2008; 17(8):1326-1335; Hefti et al. Trends Pharmacol Sci. 2006; 27(2):85-91; Mantyh et al. Anesthesiology. 2011; 115(1):189-204). In randomized clinical trials in patients with chronic pain conditions (OA and chronic low back pain), tanezumab provided clinically meaningful improvements by significantly reducing pain and improving physical function and Patient's Global Assessment (PGA) of OA (Balnescu et al. Ann Rheum Dis. 2014; 73(9):1665-1672; Brown et al. J Neurol Sci. 2014; 345(1-2):139-147; Brown et al. J Pain. 2012; 13(8):790-798; Brown et al. Arthritis Rheum. 2013; 65(7):1795-1803; Ekman et al. J Rheumatol. 2014; 41(11):2249-2259; Evans et al. J Urol. 2011; 185(5):1716-1721; Gimbel et al. Pain. 2014; 155(9):1793-1801; Katz et al. Pain. 2011; 152(10):2248-2258; Kivitz et al. Pain. 2013; 154(7):1009-1021; Lane et al. N Engl J Med. 2010; 363(16):1521-1531; Nagashima et al. Osteoarthritis Cartilage. 2011; 19(12):1405-1412; Schnitzer et al. Ann Rheum Dis. 2015; 74(6):1202-1211; Schnitzer et al. Osteoarthritis Cartilage. 2011; 19(6):639-646; Spierings et al. Pain. 2013; 154(9):1603-1612; Spierings et al. Pain. 2014; 155(11):2432-2433). During conduct of late-phase development studies, unexpected AEs requiring total joint replacement led the US Food and Drug Administration to impose a partial clinical hold on all NGF-inhibitor therapies in development (for all indications except for cancer pain). A blinded Adjudication Committee reviewed and adjudicated the joint-related AEs and determined tanezumab treatment in higher doses and in combination with NSAIDs was associated with an increase in rapidly progressive OA (Hochberg et al. Arthritis Rheumatol. 2016; 68(2):382-391). The partial clinical hold was subsequently lifted and risk-mitigation strategies have been incorporated into anti-NGF antibody trial design.

Safety is a concern regarding long-term therapy with opioids or NSAIDs. Opioids are associated with a variety of common adverse effects including somnolence, sedation, nausea, vomiting, dizziness, dry mouth, pruritus, smooth muscle spasm, urinary retention, and constipation. This is due to the presence of opioid receptors, and a role for opioid receptor signaling, in a variety of structures both within and outside of the CNS, such as the GI tract (e.g., constipation), vestibular system (e.g., nausea and dizziness), and the medulla (e.g., vomiting). Respiratory depression is a less common AE with chronic opioid use, but this potentially serious event is mediated through activation of μ-opioid receptors located in respiratory centers of the brainstem and/or structures that signal CO₂ retention to the brainstem. Finally, traditional μ-opioid receptor agonists activate dopamine signaling in the mesolimbic system by inhibiting release of GABA from inhibitory interneurons, which can produce euphoria and lead to a powerful rewarding state in some patients. Unmonitored opioid use can result in the development of addiction and it is estimated that 11.5 million people abuse opioids in the US, and deaths due to opioid overdose have risen over the past decade, with approximately 42,000 deaths per year.

### SUMMARY

The invention disclosed herein is directed to treatment chronic low back pain in patients who have a history of inadequate treatment response to prior therapy.

Accordingly, in one aspect, the invention provides a method for treating chronic low back pain in a patient, the method comprising administering to the patient an anti-nerve growth factor (NGF) antibody at a dose of about 10 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

In a further aspect, the invention provides a method for treating chronic low back pain in a patient, the method comprising administering to the patient an anti-nerve growth factor (NGF) antibody at a dose of about 5 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

In a further aspect, the invention provides a method for treating chronic low back pain in a patient, the method comprising administering to the patient an anti-nerve growth factor (NGF) antibody at a dose of about 2.5 mg to about 20 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

In some embodiments, the anti-NGF antibody is tanezumab.

Clinical benefit of the treatment according to the invention can be measured, for example, by low back pain intensity (LBPI), Roland Morris Disability Questionnaire (RMDQ) and/or Patient Global Assessment of Low Back Pain.

In some embodiments, the anti-NGF antibody effectively improves the chronic low back pain for at least 24 weeks, 32 weeks, 40 weeks, 48 weeks, or 56 weeks after start of treatment.

In some embodiments the treatment effectively reduces low back pain intensity (LBPI). In some embodiments the treatment reduces LBPI score by at least about 2.5, at least about 2.6, at least about 2.7, at least about 2.8, at least about 2.9, at least about 3.0, at least about 3.1, at least about 3.2, or at least 3.3 compared to baseline prior to or at start of treatment. In some embodiments the treatment reduces LBPI score by at least about 38-50% compared to baseline prior to or at start of treatment. In some embodiments, the treatment reduces LBPI score by at least about 38%, 39%, 40%, 41%, 42%, 43%, 44%, 45%, 46%, 47%, 48%, 49% or 50% A compared to baseline. In some embodiments the reduction in LBPI score is observed at week 16 of treatment. In some embodiments the reduction in LBPI score is observed at week 56 of treatment. In some embodiments the LBPI score is the daily average LBPI score. In some embodiments the change from baseline is the Least Squares Mean.

In some embodiments, the treatment improves low back pain as measured by Roland Morris Disability Questionnaire (RMDQ); 30% improvement in LBPI at week 16; 50% improvement in LBPI at week 16; and/or reduction in LBPI from baseline to week 2 of treatment. In some embodiments, the treatment improves RMDQ score by at least about 3.8-7.2 compared to baseline prior to or at start of treatment. In some embodiments, the treatment improves RMDQ score by at least about 5.8-7.2 compared to baseline prior to or at start of treatment. In some embodiments the treatment improves RMDQ score by at least about 6.1-6.9 compared to baseline. In some embodiments the treatment improves RMDQ score by about 6.1, 6.2, 6.3, 6.4, 6.5, 6.6, 6.7, 6.8 or 6.9 compared to baseline. In some embodiments the treatment improves RMDQ score by at least about 35-50% compared to baseline prior to or at start of treatment. In some embodiments, the treatment improves RMDQ score by at least about 35%, 36%, 37%, 38%, 39%, 40%, 41%, 42%, 43%, 44%, 45%, 46%, 47%, 48%, 49% or 50% compared to baseline. In some embodiments the improvement in RMDQ is observed at week 16 of treatment. In some embodiments the improvement in RMDQ is observed at week 56 of treatment. In some embodiments the change from baseline is the Least Squares Mean.

In some embodiments, the treatment provides 51-35% of patients with ≥50% improvement in LBPI at week 16. In some embodiments, the treatment provides 43-38% of patients with ≥50% improvement in LBPI at week 16. In some embodiments, the treatment provides at least about 43%, 44%, 45%, 46% or 47% of patients with ≥50% improvement in LBPI at week 16.

In some embodiments, the treatment improves chronic low back pain measures compared to treatment with an opioid analgesic. In some embodiments, the treatment improves chronic low back pain measures compared to treatment with tramadol. The improved chronic low back pain measures are selected from LBPI and RMDQ, as discussed above.

In some embodiments, the treatment improves chronic low back pain measures compared to treatment with NSAID. In some embodiments, the treatment improves chronic low back pain measures compared to treatment with celecoxib. The improved chronic low back pain measures are selected from LBPI and RMDQ, as discussed above.

In some embodiments the patient has a history of inadequate pain relief from or intolerance to prior therapy including analgesic therapy. The prior therapy can comprise at least three different categories of agents used for treatment of CLBP. These agents can include acetaminophen/low-dose NSAIDs; prescription NSAIDs; opioids; tapentadol; tricyclic antidepressants; benzodiazepines or skeletal muscle relaxants; lidocaine patch; and/or duloxetine or other serotonin-norepinephrine reuptake inhibitors. In some embodiments, inadequate treatment comprises the administration of an NSAID, an opioid, and at least one of the following: a tapentadol, tricyclic antidepressants, benzodiazepine or other skeletal muscle relaxants, lidocaine, and duloxetine or other serotonin-norepinephrine reuptake inhibitors. In some embodiments, the inadequate treatment comprises the administration of an NSAID, an opioid, and at least two of of the following: tapentadol, tricyclic antidepressants, benzodiazepine or other skeletal muscle relaxants, lidocaine, and duloxetine or other serotonin-norepinephrine reuptake inhibitors. In some embodiments, the inadequate treatment comprises the administration of an NSAID, an opioid, and at least three of the following: tapentadol, tricyclic antidepressants, benzodiazepine or other skeletal muscle relaxants, lidocaine, and duloxetine or other serotonin-norepinephrine reuptake inhibitors.

In some embodiments, the patient has a history of inadequate pain relief from or intolerance to at least three different classes of analgesics. The analgesic therapy can include NSAIDs, tramadol or opioids. In some embodiments, the patient has a history of inadequate pain relief from or intolerance to at least two, at least three, or at least four different classes of agents. In some embodiments, the patient has a history of inadequate pain relief from or intolerance to at least two, at least three, at least four analgesics. In some embodiments, the patient experiences some benefit from the analgesic therapy, but still requires additional pain relief. In some embodiments the patient has a history of unwillingness to take one or more analgesics, in an embodiment an opioid analgesic, in prior treatment. In some embodiments the patient was unable to take an analgesic due to contraindication. In some embodiments the patient was unable to take tramadol or opioids due to contraindication. In some embodiments the patient is diagnosed with opioid addiction. The rationale for choice of this population is to optimize the potential benefit-risk relationship for patients to be treated by selecting patients who have pain that is more severe or treatment-resistant and who have limited treatment options remaining.

In some embodiments, the patient was previously treated with the analgesic therapy prior to administering the anti-NGF antibody.

In some embodiments, the patient has a history of treatment with at least one, at least two, at least three, at least four or at least five prior therapies. The prior therapies may be from the same or different class of agent for treatment of CLBP.

In some embodiments, the patient experiences some benefit from the analgesic therapy, but still requires additional pain relief. For example, despite experiencing some benefit from an analgesic therapy, the patient continues to experience chronic low back pain, classified as Category 1 or 2 according to the classification of the Quebec Task Force in Spinal Disorders, a duration of chronic low back pain (CLBP) of ≥3 months, moderate to severe CLBP as demonstrated by an average Low Back Pain Intensity (LBPI) score of ≥5 over at least 4 daily assessments, and a baseline Patient's Global Assessment of Low Back Pain of “fair”, “poor” or “very poor”.

In some embodiments, the analgesic therapy comprises the administration of an opioid to the patient. In some embodiments the analgesic therapy comprises the administration of tramadol to the patient. In some embodiments the analgesic therapy comprises the administration of NSAID to the patient. In some embodiments the analgesic therapy comprises the administration of celecoxib to the patient.

In some embodiments, the treatment averts opioid addiction in the patient. In some embodiments, the treatment with the anti-NGF antibody avoids administration of an opioid and averts opioid addiction.

In some embodiments the patient has a history of addiction to analgesics. In some embodiments, the patient has a history of addiction to opioids. In some embodiments, the patient has a history of addiction to tramadol.

In some embodiments, the analgesic may be selected from opioids, NSAIDs, acetaminophen, In some embodiments the NSAID is selected from ibuprofen, naproxen, naprosyn, diclofenac, ketoprofen, tolmetin, slindac, mefenamic acid, meclofenamic acid, diflunisal, flufenisal, piroxim, sudoxicam, isoxicam; a COX-2 inhibitor selected from celecoxib, rofecoxib, DUP-697, flosulide, meloxicam, 6-methoxy-2 naphthylacetic acid, MK-966, nabumetone, nimesulide, NS-398, SC-5766, SC-58215, T-614; or combinations thereof. In some embodiments, the opioid may be any compound exhibiting morphine-like biological activity. In some embodiments, the opioid analgesic is selected from: tramadol, morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or combinations thereof.

In some embodiments, the patient is not administered an NSAID during the treatment with the anti-NGF antibody. In some embodiments the patient is not administered concomitant NSAID during the treatment with the anti-NGF antibody. In some embodiments the patient is not administered an NSAID for any more than 10 days in an eight week treatment interval. In some embodiments, the patient is not administered an NSAID for 16 weeks after the last dose of the antibody.

In some embodiments the patient is not administered a placebo which may be an oral placebo.

In some embodiments the patient has moderate to severe chronic low back pain.

In some embodiments, chronic low back pain is low back pain that persists for more than three consecutive months.

In some embodiments the patient has had chronic low back pain for at least 2, at least 3, at least 4, at least 5, at least 6, at least 7, at least 8, at least 9, at least 10, at least 11 or at least 12 months prior to treatment with the anti-NGF antibody. In one embodiment, the patient has had chronic low back pain for at least 3 months. In some embodiments, the patent has had chronic low back pain for at least 18, 24, 30, 36, 42, 48 or 56 months prior to treatment with the anti-NGF antibody. In some embodiments, the patient has had chronic low back pain for at least 5, at least 6, at least 7, at least 8, at least 9, at least 10, at least 11, or at least 12 years. In some embodiments, the patient has had chronic low back pain for at least 10 years.

In some embodiments, the anti-NGF antibody is administered for at least two, three, four, five, six or more doses at eight weekly intervals. In some embodiments, the anti-NGF antibody is administered to the patient for at least 16, 24, 32, 40, 48, 56, 56, 72, 80, 88 or 96 weeks. In some embodiments the anti-NGF antibody is administered to the patient for at least 80 weeks.

In some embodiments, the anti-NGF antibody is administered at a dose of 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg or 20 mg.

In some embodiments, the patient, prior to administering the anti-NGF antibody, has a) low back pain with the primary location between the 12^{th} thoracic vertebra and the lower gluteal folds, classified as Category 1 (pain without radiation) or 2 (pain with proximal radiation [above the knee]) according to the classification of the Quebec Task Force in Spinal Disorders; b) a duration of chronic low back pain of at least three months; c) a Patient Global Assessment (PGA) of low back pain measure of fair, poor, or very poor; and/or d) an average LBPI score of greater than 5 (which may be measured over at least 4 daily assessments during 5 days prior to administering the anti-NGF antibody).

In some embodiments, the patient prior to treatment with the anti-NGF antibody does not have osteoarthritis and/or pain associated with osteoarthritis.

In some embodiments, the patient prior to treatment with the anti-NGF antibody has mild radiographic evidence of knee osteoarthritis (Kellgren Lawrence Grade ≤2); and/or does not meet the American College of Rheumatology (ACR) clinical and radiographic criteria; and/or does not have pain associated with knee osteoarthritis.

In some embodiments, the patient prior to treatment with the anti-NGF antibody has no or possible radiographic evidence of hip osteoarthritis (Kellgren Lawrence Grade ≤1) and/or does not meet the American College of Rheumatology (ACR) clinical and radiographic criteria; and/or does not have pain associated with hip osteoarthritis.

In some embodiments, the patient prior to treating with the anti-NGF antibody has no symptoms and radiographic evidence of osteoarthritis of the shoulder.

In some embodiments, the patient is subjected to radiographic assessment of the knee, hip and/or shoulder prior to starting treatment with the anti-NGF antibody. In some embodiments, if radiographic assessment identified rapidly progressive osteoarthritis of the joint, the patient is excluded from the treatment with the anti-NGF antibody.

In some embodiments, the method further comprises conducting a radiographic assessment of the knee, hip and/or shoulder at regular intervals during treatment with the anti-NGF antibody.

In some embodiments, a patient may be excluded from treatment, before or during treatment, with the anti-NGF antibody if the patient has been diagnosed as having osteoarthritis of the knee or hip as defined by the American College of Rheumatology (ACR) clinical and radiographic criteria; having Kellgren-Lawrence Grade 22 radiographic evidence of hip osteoarthritis; and/or having Kellgren-Lawrence Grade 23 radiographic assessment of knee osteoarthritis and/or having symptoms and radiographic evidence of osteoarthritis of the shoulder.

In some embodiments, a patient may be excluded from treatment, before or during treatment, with the anti-NGF antibody if there is radiographic evidence of any of the following conditions as determined by the central radiology reviewer and as defined in an imaging atlas at screening: 1) rapidly progressive osteoarthritis, 2) atrophic or hypotrophic osteoarthritis, 3) subchondral insufficiency fractures, 4) spontaneous osteonecrosis of the knee (SPONK), 5) osteonecrosis, or 6) pathologic fracture.

In some embodiments, a patient may be excluded from treatment, before or during treatment, with the anti-NGF antibody if there is radiographic evidence of any of the following conditions in any screening radiograph as determined by a central radiology reviewer and as defined in an imaging atlas: excessive malalignment of the knee, severe chondrocalcinosis; other arthropathies (e.g., rheumatoid arthritis), systemic metabolic bone disease (e.g., pseudogout, Paget's disease; metastatic calcifications), large cystic lesions, primary or metastatic tumor lesions, stress or traumatic fracture.

In some embodiments, a patient may be excluded from treatment, before or during treatment, with the anti-NGF antibody if there is history or evidence of spinal disease or other conditions that could confound assessment of chronic low back pain. In some embodiments, spinal disease may include ankylosing spondylitis, rheumatoid arthritis, tumor or Paget's disease. In some embodiments conditions that could confound assessment of chronic low back pain may include fibromyalgia or back pain due to a visceral disorder.

In some embodiments, patients not having satisfactory clinical response after receiving two doses do not receive further doses.

In some embodiments, the anti-NGF antibody comprises three CDRs from the variable heavy chain region having the sequence shown in SEQ ID NO: 1 and three CDRs from the variable light chain region having the sequence shown in SEQ ID NO: 2. In some embodiments, the anti-NGF antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8. In some embodiments, the anti-NGF antibody comprises a variable heavy chain region having the sequence shown in SEQ ID NO: 1 and a variable light chain region having the sequence shown in SEQ ID NO: 2. In some embodiments, the anti-NGF antibody comprises a heavy chain having the sequence shown in SEQ ID NO: 9 and a light chain having the sequence shown in SEQ ID NO: 10. In some embodiments, the C-terminal lysine (K) of the heavy chain amino acid sequence of SEQ ID NO: 9 is optional. Thus, in some embodiments the heavy chain amino acid sequence lacks the C-terminal lysine (K) and has the sequence shown in SEQ ID NO: 11.

In some embodiments, the method further comprises administering an effective amount of a second therapeutic agent.

Also provided is an anti-NGF antibody for use in a method for treating chronic low back pain (CLBP) in a patient, as described herein.

Thus, the invention also provides an anti-NGF antibody for use in a method for treating chronic low back pain (CLBP) in a patient, the method comprising administering to the patient an anti-nerve growth factor (NGF) antibody at a dose of about 10 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

Also provided is an anti-NGF antibody for use in a method for treating chronic low back pain (CLBP) in a patient, the method comprising administering to the patient an anti-nerve growth factor (NGF) antibody at a dose of about 5 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

Also provided is an anti-NGF antibody for use in a method for treating chronic low back pain (CLBP) in a patient, the method comprising administering to the patient an anti-nerve growth factor (NGF) antibody at a dose of about 2.5 mg to about 20 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

Also provided is the use of an anti-NGF antibody in the manufacture of a medicament for use in a method for treating chronic low back pain (CLBP) in a patient, as described herein.

Thus, the invention also provides the use of an anti-NGF antibody in the manufacture of a medicament for use in a method for treating chronic low back pain (CLBP) in a patient, the method comprising administering to the patient an anti-nerve growth factor (NGF) antibody at a dose of about 10 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

Also provided is the use of an anti-NGF antibody in the manufacture of a medicament for use in a method for treating chronic low back pain (CLBP) in a patient, the method comprising administering to the patient an anti-nerve growth factor (NGF) antibody at a dose of about 5 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

Also provided is the use of an anti-NGF antibody in the manufacture of a medicament for use in a method for treating chronic low back pain (CLBP) in a patient, the method comprising administering to the patient an anti-nerve growth factor (NGF) antibody at a dose of about 2.5 mg to about 20 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

In embodiments that refer to a method of treating chronic low back pain (CLBP) as described herein, such embodiments are also further embodiments of an anti-NGF antibody for use in that treatment, or alternatively of the use of an anti-NGF antibody in the manufacture of a medicament for use in that treatment.

Preferred features of each aspect of the invention apply equally to each other aspect mutatis mutandis.

### BRIEF DESCRIPTION OF THE FIGURES/DRAWINGS

FIG. 1 is a study outline for the study described in Example 1.

FIG. 2 shows the change in LBPI and RMDQ scores from baseline to week 16

FIG. 3 shows change from baseline for LBPI score up to week 56 for the study described in Example 1.

FIG. 4 shows change from baseline for RMDQ up to week 56 for the study described in Example 1.

FIG. 5 shows the change in both LPBI and RMDQ scores throughout the 56 week treatment period.

FIG. 6 shows the change in LBPI and RMDQ scores from baseline to week 56.

FIG. 7 shows the proportion of patients with a >0% to ≥90% improvement in LBPI at week 16.

FIG. 8 is a study outline for the study described in Example 2.

FIG. 9 shows the change in LBPI scores from baseline to week 56 for the study described in Example 2.

FIG. 10 shows the change from baseline for RMDQ up to week 56 for the study described in Example 2.

### DETAILED DESCRIPTION

The invention disclosed herein is directed to treatment of chronic low back pain in patients who have a history of inadequate treatment response to prior therapy including analgesics.

Accordingly, in one aspect, the invention provides a method for treating chronic low back pain in a patient, the method comprising administering to the patient an anti-nerve growth factor (NGF) antibody at a dose of about 10 mg every 8 weeks via subcutaneous injection; wherein the patient has a history of inadequate treatment response to prior therapy including analgesics and the treatment with the anti-NGF antibody effectively improves chronic low back pain at least 16 weeks after start of treatment with the anti-NGF antibody.

In a further aspect, the invention provides a method for treating chronic low back pain in a patient, the method comprising administering to the patient an anti-nerve growth factor (NGF) antibody at a dose of about 5 mg every 8 weeks via subcutaneous injection; wherein the patient has a history of inadequate treatment response to prior therapy including analgesics and the treatment with the anti-NGF antibody effectively improves chronic low back pain at least 16 weeks after start of treatment with the anti-NGF antibody.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings:

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxi bodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

In some instances, Fv framework region (FR) residues of the human immunoglobulin are replaced by corresponding non-human residues. Furthermore, the humanized antibody may include residues that are found neither in the recipient antibody nor in the imported CDR or framework sequences, but are included to further refine and optimize antibody performance. In general, the humanized antibody will include substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will include at least a portion of an immunoglobulin constant region or domain (Fc), typically that of a human immunoglobulin. In some aspects of the invention the antibodies have Fc regions modified as described in PCT International Publication No. WO 99/58572. Other forms of humanized antibodies have one or more CDRs (CDR L1, CDR L2, CDR L3, CDR H1, CDR H2, or CDR H3) which may be altered with respect to the original antibody, which are also termed one or more CDRs “derived from” one or more CDRs from the original antibody.

Humanization can be essentially performed following the method of Winter and co-workers (Jones et al. Nature 321:522-525 (1986); Riechmann et al. Nature 332:323-327 (1988); Verhoeyen et al. Science 239:1534-1536 (1988)), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205; which are incorporated herein by reference in its entirety. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may include residues that are not found in the recipient antibody or in the donor antibody. These modifications are made to further refine antibody performance (e.g., to obtain desired affinity). In general, the humanized antibody will include substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will include at least a portion of an immunoglobulin constant region (Fc), typically that of a human immunoglobulin. For further details see Jones et al. Nature 321:522-525 (1986); Riechmann et al. Nature 332:323-327(1988); and Presta Curr. Op. Struct. Biol. 2:593-596 (1992); which are incorporated herein by reference in its entirety. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and PCT International Publication No. WO 01/27160, where humanized antibodies and techniques for producing humanized antibodies having improved affinity for a predetermined antigen are disclosed.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The antibody “tanezumab” is a humanized immunoglobulin G Type 2 (IgG2) monoclonal antibody directed against human nerve growth factor (NGF). Tanezumab binds to human NGF with high affinity and specificity and blocks the activity of NGF effectively in cell culture models. Tanezumab and/or its murine precursor have been shown to be an effective analgesic in animal models of pathological pain including arthritis, cancer pain, and post-surgical pain. Tanezumab has the sequences for the variable heavy chain region and variable light chain region of SEQ ID Nos: 1 and 2, respectively. The heavy chain and light chain sequences are provided in SEQ ID NO: 9 and 10, or SEQ ID NOs: 11 and 10. The C-terminal lysine (K) of the heavy chain amino acid sequence of SEQ ID NO: 9 is optional and may be processed, resulting in a heavy chain amino acid sequence lacking the C-terminal lysine (K) and having the sequence shown in SEQ ID NO: 11. Sequences of tanezumab are provided in Table 1 below. Tanezumab is described, as antibody E3, in WO2004058184, herein incorporated by reference.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a target (e.g., PD-1) epitope is an antibody that binds this epitope with greater affinity, avidity, more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g. B cell receptor), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

As used herein, “treatment” is an approach for obtaining beneficial or desired clinical results. For purposes of this invention, beneficial or desired clinical results include reduction or improvement in chronic low back pain, for example as compared to before administration of the anti-NGF antibody.

“Ameliorating” means a lessening or improvement of chronic low back pain, for example as compared to not administering an anti-NGF antibody as described herein. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates chronic low back pain. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing chronic low back pain, decreasing the dose of other medications required to treat the disease, enhancing the effect of another medication, and/or delaying the progression of the disease in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The term “inadequate treatment response to prior therapy” refers to a patient who has experienced an adverse event after treatment with the prior therapy; who is refractory to treatment with the prior therapy; who shows no clinically meaningful improvement in one or more measures of chronic low back pain with prior therapy; who experiences some benefit from prior therapy but still requires additional pain relief; who is addicted to the prior therapy (including analgesics such as opioids); and/or who is unwilling to take the prior therapy. In some embodiments, the patient has a history of inadequate pain relief from or intolerance to prior therapy, which may comprise at least three different classes of analgesics.

Treatment “effectively improves” or “effectively reduces” when assessment of the chronic low back pain is quantified via a clinical measure relative to baseline and during and/or after the treatment period. The difference between the clinical measure at baseline and during/after treatment is compared and used to determine whether the low back pain has improved and the treatment is effective. This comparison can include comparison to placebo or to one or more of the prior therapies. In one embodiment, the comparison can be to placebo or to treatment with an opioid analgesic, such as tramadol; or a NSAID, such as celecoxib. The clinical measure can be Low Back Back Intensity (LBPI). The Low Back Pain Intensity (LBPI) measure can be determined for the patient at baseline and then determined throughout the treatment period, such as at weeks 2, 4, 6, 8, 16, 24, 32, 40, 48, 56, or longer. Similarly, the Roland Morris Disability Questionnaire (RMDQ) can also be determined in this manner. Yet further, the Patient Global Assessment (PGA) of low back pain measure can also be determined in this manner.

In some embodiments the treatment effectively reduces low back pain intensity (LBPI). In some embodiments the treatment reduces LBPI score by at least about 2.5, at least about 2.6, at least about 2.7, at least about 2.8, at least about 2.9, at least about 3, at least about 3.1, at least about 3.2, or at least about 3.3 compared to baseline prior to or at start of treatment. In some embodiments the treatment reduces LBPI score by at least about 38-50% compared to baseline prior to or at start of treatment. In some embodiments, the treatment reduces LBPI score by at least about 38%, 39%, 40%, 41%, 42%, 43%, 44%, 45%, 46%, 47%, 48%, 49%, 50%, 55%, 60%, 70%, 75%, 80%, 85%, 90% or 95% compared to baseline. In some embodiments the treatment effectively reduces LBPI score compared to placebo. In some embodiments the treatment effectively reduces LBPI score by at least about 0.2, 0.25, 0.3, 0.35, 0.4, 0.45, 0.5, 0.55, 0.6 or 0.65 compared to placebo. In some embodiments the treatment effectively reduces LBPI score compared to baseline and/or placebo to a greater extent than an opioid analgesic, which may be tramadol and/or a NSAID, which may be celecoxib. In some embodiments the treatment reduces LBPI score by at least about 2-10% more than placebo and/or an opioid analgesic, which may be tramadol. In some embodiments the treatment reduces LBPI score by at least about 2%, 3%, 4%, 5%, 6%, 7%, 8%, 9%, 10%, 15%, 20%, 25%, 30%, 35%, 40%, 45% or 50% more than placebo and/or an opioid analgesic, which may be tramadol. In some embodiments the reduction in LBPI is observed at week 16 of treatment. In some embodiments the reduction in LBPI is observed at week 56 of treatment. In some embodiments the LBPI score is the daily average LBPI score. In some embodiments the change from baseline is the Least Squares Mean.

In some embodiments, the treatment improves RMDQ score by at least about 3.8-7.2 compared to baseline prior to or at start of treatment. In some embodiments, the treatment improves RMDQ score by at least about 5.8-7.2 compared to baseline prior to or at start of treatment. In some embodiments the treatment improves RMDQ score by at least about 6.1-6.9 compared to baseline. In some embodiments the treatment improves RMDQ score by about 6.1, 6.2, 6.3, 6.4, 6.5, 6.6, 6.7, 6.8 or 6.9 compared to baseline. In some embodiments the treatment improves RMDQ score by at least about 35-50% compared to baseline prior to or at start of treatment. In some embodiments, the treatment improves RMDQ score by at least about 35%, 36%, 37%, 38%, 39%, 40%, 41%, 42%, 43%, 44%, 45%, 46%, 47%, 48%, 49%, 50%, 55%, 60%, 70%, 75%, 80%, 85%, 90% or 95% compared to baseline. In some embodiments the treatment effectively improves RMDQ score compared to placebo. In some embodiments the treatment effectively improves RMDQ score by at least about 0.7, 0.8, 0.9, 1.0, 1.1, 1.2, 1.3, 1.4, 1.5, 1.6, 1.7, 1.8, 1.9, 2.0, 2.1, 2.2, 2.3 or 2.4 compared to placebo. In some embodiments the treatment effectively improves RMDQ score compared to baseline and/or placebo to a greater extent than an opioid analgesic, which may be tramadol. In some embodiments the treatment improves RMDQ score by at least about 2-10% more than placebo and/or an opioid analgesic, which may be tramadol. In some embodiments the treatment improves RMDQ score by at least about 2%, 3%, 4%, 5%, 6%, 7%, 8%, 9%, 10%, 15%, 20%, 25%, 30%, 35%, 40%, 45% or 50% more than placebo and/or an opioid analgesic, which may be tramadol. In some embodiments the improvement in RMDQ is observed at week 16 of treatment. In some embodiments the improvement in RMDQ is observed at week 56 of treatment. In some embodiments the change from baseline is the Least Squares Mean

In some embodiments, the treatment provides 51-35% of patients with ≥50% improvement in LBPI at week 16. In some embodiments, the treatment effectively provides 43-48% of patients with ≥50% improvement in LBPI at week 16. In some embodiments, the treatment provides at least about 43%, 44%, 45%, 46% or 47% of patients with ≥50% improvement in LBPI at week 16. In some embodiments the treatment effectively provides the proportion of patients with ≥50% improvement in LBPI at week 16 compared to placebo. In some embodiments the treatment effectively improves the proportion of patients with ≥50% improvement in LBPI at week 16 by an odds ratio of at least about 1.25, 1.30, 1.35, 1.40, 1.45, 1.50, 1.55 or 1.60 compared to placebo. In some embodiments the treatment effectively improves the proportion of patients with ≥50% improvement in LBPI compared to baseline and/or placebo to a greater extent than an opioid analgesic, which may be tramadol. In some embodiments the improvement is observed at week 16 of treatment. In some embodiments the improvement is observed at week 56 of treatment.

In some embodiments, the treatment effectively improves the proportion of patients with ≥30% improvement in LBPI at week 16. In some embodiments, the treatment effectively provides at least 58% of patients with ≥30% improvement in LBPI at week 16. In some embodiments, the treatment provides at least about 59%, 60%, 61%, 62%, 63%, 64%, 65%, 66%, 67%, 68%, 69% or 70% of patients with ≥30% improvement in LBPI at week 16. In some embodiments the treatment effectively provides the proportion of patients with ≥30% improvement in LBPI at week 16 compared to placebo. In some embodiments the treatment effectively improves the proportion of patients with ≥30% improvement in LBPI at week 16 by an odds ratio of at least about 1.25, 1.30, 1.35, 1.40, 1.45, 1.50, 1.55 or 1.60 compared to placebo. In some embodiments the treatment effectively improves the proportion of patients with ≥30% improvement in LBPI compared to baseline and/or placebo to a greater extent than an opioid analgesic, which may be tramadol. In some embodiments the improvement is observed at week 16 of treatment. In some embodiments the improvement is observed at week 56 of treatment.

In some embodiments, the treatment effectively improves LBPI score at week 2 by at least about 1.3, 1.4, 1.5, 1.6, 1.7 or 1.8 compared to baseline prior to or at start of treatment. In some embodiments the treatment improves LBPI score at week 2 by at least about 15-30% compared to baseline prior to or at start of treatment. In some embodiments the treatment effectively improves LBPI score at week 2 by at least about 0.2, 0.3, 0.4, 0.5 or 0.6 compared to placebo. In some embodiments, the treatment improves LBPI score at week 2 by at least about 3-15% more than placebo. In some embodiments, the treatment effectively improves LBPI score at week 2 compared to baseline and/or placebo to a greater extent than an opioid analgesic, which may be tramadol. In some embodiments the change from baseline is the Least Squares Mean.

In some embodiments the treatment effectively improves LBPI and/or RMDQ score at week 56 of treatment compared to baseline prior to or at start of treatment. In some embodiments, the treatment effectively improves LBPI and/or RMBQ score at week 56 more than an opioid analgesic, which may be tramadol. In some embodiments, the treatment effectively improves LBPI and/or RMBQ score at week 56 more than a NSAID analgesic, which may be celecoxib.

The term “baseline” refers to a value of a low back pain associated measure for a patient prior to administration of the anti-NGF antibody as part of the treatment method. In some embodiments, the term “baseline” refers to a value of a sign or symptom associated measure for control healthy subjects that do not have chronic low back pain.

In some embodiments, treatment with the anti-NGF antibody effectively improves chronic low back pain at at least 8 weeks after start of treatment with the antibody. In some embodiments, treatment with the anti-NGF antibody effectively improves chronic low back pain at at least 10 weeks after start of treatment with the antibody. In some embodiments, treatment with the anti-NGF antibody effectively improves chronic low back pain at at least 12 weeks after start of treatment with the antibody. In some embodiments, treatment with the anti-NGF antibody effectively improves chronic low back pain at at least 14 weeks after start of treatment with the antibody. In some embodiments, treatment with the anti-NGF antibody effectively improves chronic low back pain at at least 16 weeks after start of treatment with the antibody. In some embodiments, treatment with the anti-NGF antibody effectively improves chronic low back pain at at least 24 weeks after start of treatment with the antibody. In some embodiments, treatment with the anti-NGF antibody effectively improves chronic low back pain at at least 32 weeks after start of treatment with the antibody. In some embodiments, treatment with the anti-NGF antibody effectively improves chronic low back pain at at least 40 weeks after start of treatment with the antibody. In some embodiments, treatment with the anti-NGF antibody effectively improves chronic low back pain at at least 56 weeks after start of treatment with the antibody.

In some embodiments, the chronic low back pain is moderate to severe.

The Low Back Pain Intensity (LBPI) measure is assessed with an 11-point numeric rating scale ranging from 0 (no pain) to 10 (worst possible pain). The LBPI score can be the daily average LBPI score.

The Roland Morris Disability Questionnaire (RMDQ) is an index of how well subjects with low back pain are able to function with regard to daily activities (Roland M, Fairbank. The Roland-Morris Questionnaire and the Oswestry Disability Questionnaire. Spine. 2000; 25 (4)3115-3124). It is a low back pain-specific assessment of physical function with scores ranging from 0 to 24 (lower scores indicate better function).

The Patient Global Assessment (PGA) measure is a global evaluation that utilizes a 5-point Likert scale with a score of 1 being best (very good) and a score of 5 being worst (very poor). In this assessment, a patient answers the following question: “Considering all the ways your low back pain affects you, how are you doing today?”

| Grade | Description |
|---|---|
| 1 - Very Good | Asymptomatic and no limitation of normal activities |
| 2 - Good | Mild symptoms and no limitation of normal activities |
| 3 - Fair | Moderate symptoms and limitation of some normal |
| | activities |
| 4 - Poor | Severe symptoms and inability to carry out most |
| | normal activities |
| 5 - Very Poor | Very severe symptoms which are intolerable and |
| | inability to carry out all normal activities |

Kellgren-Lawrence x-ray grade is a method of classifying the severity of osteoarthritis (Kellgren and Lawrence., Ann Rheum Dis 2000:16(4): 494-502).

The American College of Rheumatology (ACR) classification criteria for osteoarthritis (Altman, et al. Arthritis Rheum 1986; 29:1039-49) includes clinical and radiographic criteria for osteoarthritis of the hip or knee.

Rapidly progressive osteoarthritis (RPOA) of the hip was first described by Forestier in 1957 and subsequently described in a number of studies as atrophic osteoarthritis, rapidly destructive osteoarthritis, rapidly destructive arthropathy, rapidly progressive hip disease, or rapidly destructive coxarthrosis. Rapidly progressive hip osteoarthritis is characterized by subjects who typically present with hip pain, often severe, with radiographs that show rapid joint space narrowing as a result of chrondrolysis from a prior radiograph and, subsequently, an osteolytic phase with severe progressive atrophic bone destruction involving the femoral head and the acetabulum. There can be marked flattening of the femoral head and loss of subchondral bone in the weight bearing area and in some cases the femoral head appears sheared off. Osteophytes are typically conspicuously small or absent. Bone sclerosis is often present at sites of impaction of the femoral head and the acetabulum, subchondral detritus is invariably present and bone fragmentation and debris are commonly observed that can lead to synovitis. Lequesne proposed that subjects with 2 mm/year or greater of joint space narrowing or loss of more than 50% of the joint space within 1 year should be considered to have rapidly progressive osteoarthritis. Due to a lack of longitudinal studies, it is not clear what proportion of subjects with rapid loss of joint space (chondrolysis) will progress to have bone destruction. Rapid progression of osteoarthritis has also been described in the shoulder and the knee.

The incidence of rapidly progressive osteoarthritis in the overall osteoarthritis population is not well defined. For rapid progression of hip osteoarthritis, the prevalence ranges from approximately 2% to 18% based on clinical case series analyses. The pathophysiology of rapidly progressive osteoarthritis is not understood. Various mechanisms have been proposed including; ischemia, venous stasis, local nutritional deficiencies, synovitis, mechanical overloading, NSAID or corticosteroid use, intra articular deposition of hydroxyapatite or pyrophosphate crystals and subchondral insufficiency fractures.

There is a lack of data in the literature on the rate of rapidly progressive OA in a progressed OA population and the causes of this disease progression. As described by Hochberg et al (Arthritis Rheumatol., vol. 68, no. 2. pp. 382-391). “Rapidly progressive osteoarthritis is characterized by pain, with radiographs showing rapid joint space narrowing as a result of chondrolysis (type-1).” Possibly subsequently, these patients progress to an osteolytic phase with severe progressive atrophic bone destruction (type-2). However, this continuity is not clear due to a lack of longitudinal studies (Hochberg et al., Arthritis Rheumatol., vol. 68, no. 2. pp. 382-391).

Radiographic assessments (x-rays) of both knees, both hips and both shoulders can be performed or obtained prior to treatment, at screening. Other major joints exhibiting signs or symptoms suggestive of osteoarthritis may also be imaged. A major joint is defined as a mobile synovial joint in the limbs such as shoulders, elbows, wrists, hips, knees, ankles and excluding the joints of the toes and hands. Any joint imaged at Screening or other at risk joints identified during the study period should also be imaged.

A central radiology reader (Central Reader) may review the radiology images for assessment of eligibility including determination and identification of exclusionary joint conditions. Radiographs required at screening may be obtained at least two weeks prior to the beginning of the Initial Pain Assessment Period (IPAP) to permit central radiology review of the images and to establish subject eligibility for initial dosing with an NGF antibody. In some embodiments, subjects may not be permitted to start dosing with an NGF antibody until the screening radiographs are reviewed and eligibility is established.

The X-ray technologists, in addition to their professional training and certifications, are trained in performing the radiographic protocols for the knees, hips, and shoulders. To facilitate reproducibility and accuracy of joint space width measurement in the knees and hips, a semi-automated software and positioning frame standardized subject and joint positioning protocol can be utilized. The Core Imaging Laboratory may be responsible for working with the sites to ensure quality, standardization and reproducibility of the radiographic images/assessments made at the Screening and follow-up time-points. Additional details regarding the required X-rays may be provided in a site imaging manual.

Central radiology readers (Central Readers) may be board certified radiologists or have the international equivalent as musculoskeletal radiologists. The Central Readers may be governed by an imaging atlas and an imaging Charter which includes a specific description of the scope of their responsibilities. Central Readers may review the radiology images at Screening for assessment of eligibility (including determination of Kellgren-Lawrence Grade) and identification of exclusionary joint conditions such as rapidly progressive osteoarthritis, atrophic or hypotrophic osteoarthritis, subchondral insufficiency fractures (spontaneous osteonecrosis of the knee [SPONK]), primary osteonecrosis and pathological fractures. After start of treatment, the Central Reader may review radiology images for diagnosis of joint conditions that would warrant further evaluation by the Adjudication Committee such as possible or probable rapidly progressive osteoarthritis, subchondral insufficiency fractures (spontaneous osteonecrosis of the knee [SPONK]), primary osteonecrosis or pathological fracture.

For subjects who are identified with a possible or probable joint event (i.e., rapidly progressive osteoarthritis, subchondral insufficiency fractures, spontaneous osteonecrosis of the knee (SPONK), primary osteonecrosis or pathological fracture) and subjects undergoing total joint replacement for any reason, all images and other source documentation may be provided to the blinded Adjudication Committee for review and adjudication of the event. The Adjudication Committee's assessment of the event may represent the final classification of the event.

Patients may be excluded from treatment with the anti-NGF antibody, during or before treatment with the anti-NGF antibody, if there is radiographic evidence of any of the following conditions in any screening radiograph as determined by a central radiology reviewer and as defined in an imaging atlas: excessive malalignment of the knee, severe chondrocalcinosis; other arthropathies (e.g., rheumatoid arthritis), systemic metabolic bone disease (e.g., pseudogout, Paget's disease; metastatic calcifications), large cystic lesions, primary or metastatic tumor lesions, stress or traumatic fracture. In some embodiments a patient may be excluded from treatment with the anti-NGF antibody, before or during the treatment with the anti-NGF antibody, if there is radiographic evidence of any of the following conditions as determined by the central radiology reviewer and as defined in an imaging atlas at screening: 1) rapidly progressive osteoarthritis, 2) atrophic or hypotrophic osteoarthritis, 3) subchondral insufficiency fractures, 4) spontaneous osteonecrosis of the knee (SPONK), 5) osteonecrosis, or 6) pathologic fracture.

In some embodiments a patient may be excluded from treatment, before or during treatment, with the anti-NGF antibody if the patient has been diagnosed as having osteoarthritis of the knee or hip as defined by the American College of Rheumatology (ACR) clinical and radiographic criteria; having Kellgren-Lawrence Grade 22 radiographic evidence of hip osteoarthritis; and/or having Kellgren-Lawrence Grade 23 radiographic assessment of knee osteoarthritis and/or having symptoms and radiographic evidence of osteoarthritis of the shoulder. The radiographic criteria may be assessed by a Central Reader.

A “patient”, an “individual” or a “subject”, used interchangeably herein, is a mammal, more preferably, a human. Mammals also include, but are not limited to, farm animals (e.g., cows, pigs, horses, chickens, etc.), sport animals, pets, primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “effector function” refers to the biological activities attributable to the Fc region of an antibody. Examples of antibody effector functions include, but are not limited to, antibody-dependent cell-mediated cytotoxicity (ADCC), Fc receptor binding, complement dependent cytotoxicity (CDC), phagocytosis, C1q binding, and down regulation of cell surface receptors (e.g., B cell receptor; BCR). See, e.g., U.S. Pat. No. 6,737,056. Such effector functions generally require the Fc region to be combined with a binding domain (e.g., an antibody variable domain) and can be assessed using various assays known in the art for evaluating such antibody effector functions. An exemplary measurement of effector function is through Fcγ3 and/or C1q binding.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” or “CDC” refers to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202: 163 (1996), may be performed.

The term “kₒₙ” or “kₐ”, as used herein, refers to the rate constant for association of an antibody to an antigen. Specifically, the rate constants (kₒₙ or kₐ and k_{off} or k_{d}) and equilibrium dissociation constants are measured using whole antibody (i.e. bivalent) and monomeric proteins.

The term “k_{off}” or “k_{d}”, as used herein, refers to the rate constant for dissociation of an antibody from the antibody/antigen complex.

The term “K_{D}”, as used herein, refers to the equilibrium dissociation constant of an antibody-antigen interaction.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The term “subcutaneous administration” refers to the administration of a substance into the subcutaneous layer.

The term “preventing” or “prevent” refers to (a) keeping a disorder from occurring or (b) delaying the onset of a disorder or onset of symptoms of a disorder.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

### Anti-NGF Antibodies

Provided herein are anti-NGF antibodies for use in the methods of treatment as described herein.

In one aspect, the anti-NGF antibody binds to NGF and inhibits binding of NGF to trkA and/or p75.

In an embodiment, the antibody comprises three CDRs from the heavy chain variable region of SEQ ID NO: 1. In some embodiments, the antibody comprises three CDRs from the light chain variable region of SEQ ID NO: 2. In some embodiments the antibody comprises three CDRs from the heavy chain variable region of SEQ ID NO: 1 and three CDRs from the light chain variable region of SEQ ID NO: 2.

In some embodiments, the CDRs may be defined in accordance with any of Kabat, Chothia, extended, AbM, contact, and/or conformational definitions. In some embodiments, the CDRS shown in SEQ ID NO:3, SEQ ID NO:4. SEQ ID NO:5. SEQ ID NO:6, SEQ ID NO:7, and SEQ ID NO:8 are determined by a combination of the Kabat and Chothia methods.

Exemplary antibody sequences used for the present invention include, but are not limited to, the sequences listed below.

TABLE 1

| SEQ ID NO: | Sequence |
|---|---|
| 1 | Variable heavy chain region: |
| | QVQLQESGPGLVKPSETLSLTCTVSGFSLIGYDLNWIRQPPGKGLEW |
| | IGIIWGDGTTDYNSAVKSRVTISKDTSKNQFSLKLSSVTAADTAVYYC |
| | ARGGYWYATSYYFDYWGQGTLVTVS |
| 2 | Variable light chain region: |
| | DIQMTQSPSSLSASVGDRVTITCRASQSISNNLNWYQQKPGKAPKLL |
| | IYYTSRFHSGVPSRFSGSGSGTDFTFTISSLQPEDIATYYCQQEHTLP |
| | YTFGQGTKLEIKRT |
| 3 | Extended HCDR1: |
| | GFSLIGYDLN |
| 4 | Extended HCDR2: |
| | IIWGDGTTDYNSAVKS |
| 5 | Extended HCDR3: |
| | GGYWYATSYYFDY |
| 6 | Extended LCDR1: |
| | RASQSISNNLN |
| 7 | Extended LCDR2: |
| | YTSRFHS |
| 8 | Extended LCDR3: |
| | QQEHTLPYT |
| 9 | Heavy chain*: |
| | QVQLQESGPGLVKPSETLSLTCTVSGFSLIGYDLNWIRQPPGKGLEW |
| | IGIIWGDGTTDYNSAVKSRVTISKDTSKNQFSLKLSSVTAADTAVYYC |
| | ARGGYWYATSYYFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSNFGTQTYTCNVDHKPSNTKVDKTVERKCCVECPPCPAP |
| | PVAGPSVFLFPPKPKDTLMISRTPEVTCVVVDVSHEDPEVQFNWYV |
| | DGVEVHNAKTKPREEQFNSTFRVVSVLTVVHQDWLNGKEYKCKVS |
| | NKGLPSSIEKTISKTKGQPREPQVYTLPPSREEMTKNQVSLTCLVKG |
| | FYPSDIAVEWESNGQPENNYKTTPPMLDSDGSFFLYSKLTVDKSRW |
| | QQGNVFSCSVMHEALHNHYTQKSLSLSPGK |
| 10 | Light chain: |
| | DIQMTQSPSSLSASVGDRVTITCRASQSISNNLNWYQQKPGKAPKLL |
| | IYYTSRFHSGVPSRFSGSGSGTDFTFTISSLQPEDIATYYCQQEHTLP |
| | YTFGQGTKLEIKRTVAAPSVFIFPPSDEQLKSGTASVVCLLNNFYPRE |
| | AKVQWKVDNALQSGNSQESVTEQDSKDSTYSLSSTLTLSKADYEKH |
| | KVYACEVTHQGLSSPVTKSFNRGEC |
| 11 | Heavy chain (C-terminal lysine (K) processed) |
| | QVQLQESGPGLVKPSETLSLTCTVSGFSLIGYDLNWIRQPPGKGLEW |
| | IGIIWGDGTTDYNSAVKSRVTISKDTSKNQFSLKLSSVTAADTAVYYC |
| | ARGGYWYATSYYFDYWGQGTLVTVSSASTKGPSVFPLAPCSRSTS |
| | ESTAALGCLVKDYFPEPVTVSWNSGALTSGVHTFPAVLQSSGLYSLS |
| | SVVTVPSSNFGTQTYTCNVDHKPSNTKVDKTVERKCCVECPPCPAP |
| | PVAGPSVFLFPPKPKDTLMISRTPEVTCVVVDVSHEDPEVQFNWYV |
| | DGVEVHNAKTKPREEQFNSTFRVVSVLTVVHQDWLNGKEYKCKVS |
| | NKGLPSSIEKTISKTKGQPREPQVYTLPPSREEMTKNQVSLTCLVKG |
| | FYPSDIAVEWESNGQPENNYKTTPPMLDSDGSFFLYSKLTVDKSRW |
| | QQGNVFSCSVMHEALHNHYTQKSLSLSPG |

[* C-terminal lysine (K) of the heavy chain amino acid sequence of SEQ ID NO: 9 is optional]

In one embodiment, the antibody is tanezumab.

In some embodiments, the antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

In some embodiments, the antibody comprises a heavy chain variable region (VH) having the sequence shown in SEQ ID NO: 1. In some embodiments, the antibody comprises a light chain variable region (VL) having the amino acid sequence of SEQ ID NO: 2. In some embodiments, the antibody comprises a heavy chain variable region (VH) having the sequence shown in SEQ ID NO: 1 and a light chain variable region (VL) having the amino acid sequence of SEQ ID NO: 2.

In some embodiments, the antibody comprises a heavy chain having the amino acid sequence shown in SEQ ID NO: 9 and a light chain having the amino acid sequence shown in SEQ ID NO: 10. In some embodiments, the C-terminal lysine (K) of the heavy chain amino acid sequence of SEQ ID NO: 9 is optional. Thus, in some embodiments the heavy chain amino acid sequence lacks the C-terminal lysine (K) and has the sequence shown in SEQ ID NO: 11. Thus, in some embodiments, the antibody comprises a heavy chain having the amino acid sequence shown in SEQ ID NO: 11 and a light chain having the amino acid sequence shown in SEQ ID NO: 10.

In some embodiments, the antibody is fasinumab or REGN475 (see, for example, US 2009/0041717, herein incorporated by reference) or has the same or substantially the same amino acid sequence as fasinumab or REGN475. In some embodiments, the antibody is fulranumab.

The antibodies as described herein can be made by any method known in the art. An antibody may be made recombinantly using a suitable host cell. A nucleic acid encoding an anti-NGF antibody of the present disclosure can be cloned into an expression vector, which can then be introduced into a host cell, where the cell does not otherwise produce an immunoglobulin protein, to obtain the synthesis of an antibody in the recombinant host cell. Any host cell susceptible to cell culture, and to expression of protein or polypeptides, may be utilized in accordance with the present invention. In certain embodiments, the host cell is mammalian. Mammalian cell lines available as hosts for expression are well known in the art and include many immortalized cell lines available from the American Type Culture Collection (ATCC). Nonlimiting exemplary mammalian cells include, but are not limited to, NS0 cells, HEK 293 and Chinese hamster ovary (CHO) cells, and their derivatives, such as 293-6E and CHO DG44 cells, CHO DXB11, and Potelligent® CHOK1SV cells (BioWa/Lonza, Allendale, N.J.). Mammalian host cells also include, but are not limited to, human cervical carcinoma cells (HeLa, ATCC CCL 2), baby hamster kidney (BHK, ATCC CCL 10) cells, monkey kidney cells (COS), and human hepatocellular carcinoma cells (e.g., Hep G2). Other non-limiting examples of mammalian cells that may be used in accordance with the present invention include human retinoblasts (PER.C6®; CruCell, Leiden, The Netherlands); monkey kidney CV1 line transformed by SV40 (COS-7, ATCC CRL 1651); human embryonic kidney line 293 (HEK 293) or 293 cells subcloned for growth in suspension culture (Graham et al., J. Gen Virol. 1997; 36:59); mouse sertoli cells (TM4, Mather, Biol. Reprod. 1980; 23:243-251); monkey kidney cells (CV1 ATCC CCL 70); African green monkey kidney cells (VERO-76, ATCC CRL-1 587); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 1982; 383:44-68); MRC 5 cells; FS4 cells; a human hepatoma line (Hep G2); and numerous myeloma cell lines, including, but not limited to, BALB/c mouse myeloma line (NS0/1, ECACC No: 85110503), NS0 cells and Sp2/0 cells.

Additionally, any number of commercially and non-commercially available cell lines that express polypeptides or proteins may be utilized. One skilled in the art will appreciate that different cell lines might have different nutrition requirements and/or might require different culture conditions for optimal growth and polypeptide or protein expression and will be able to modify conditions as needed.

For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies.

Hybridomas that produce antibodies used for the present invention may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target (e.g., PD-1), a human target protein (e.g., PD-1), or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., J. Immunol. Methods 329, 112, 2008; U.S. Pat. No. 7,314,622.

In some embodiments, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

In some embodiments, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

### Polynucleotides, Vectors, and Host Cells

The invention also provides polynucleotides encoding any of the anti-NGF antibodies as described herein. In one aspect, the invention provides a method of making any of the polynucleotides described herein. Polynucleotides can be made and expressed by procedures known in the art.

In another aspect, the invention provides compositions (such as a pharmaceutical compositions) comprising any of the polynucleotides of the invention. In some embodiments, the composition comprises an expression vector comprising a polynucleotide encoding any of the anti-NGF antibodies described herein.

In another aspect, provided is an isolated cell line that produces the anti-NGF antibodies as described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficol/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to NGF is effected by an immunoassay or FACS. A cell overexpressing the antibody or protein of interest can be identified.

### Compositions

The invention also provides pharmaceutical compositions comprising an effective amount of an anti-NGF antibody as described herein, and such pharmaceutical compositions for use in methods of treatment as described herein. Examples of such compositions, as well as how to formulate, are also described herein.

It is understood that the compositions can comprise more than one anti-NGF antibody.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The anti-NGF antibody, and compositions thereof, can also be used in conjunction with, or administered separately, simultaneously, or sequentially with other agents that serve to enhance and/or complement the effectiveness of the agents.

### Methods for Treating Chronic Low Back Pain

In one aspect, the invention provides a method for treating chronic low back pain (CLBP) in a patient as defined herein.

In some embodiments, the methods described herein further comprise a step of treating a subject with an additional form of therapy. In some embodiments, the additional form of therapy is an additional therapeutic agent which may be selected from an NGF antagonist, a trkA antagonist, an IL-1 antagonist, an IL-6 antagonist, an IL-6R antagonist, an opioid, acetaminophen, a local anesthetic, an NMDA modulator, a cannabinoid receptor agonist, a P2X family modulator, a VR1 antagonist, a substance P antagonist, a Nav1.7 antagonist, a cytokine or cytokine receptor antagonist, a steroid, other inflammatory inhibitors and a corticosteroid.

In some embodiments, the method described herein does not comprise administration of an NSAID to the patient. In some embodiments, the method described herein does not comprise administration of an opioid to the patient.

With respect to all methods described herein, reference to anti-NGF antibodies also includes compositions comprising one or more additional agents. These compositions may further comprise suitable excipients, such as pharmaceutically acceptable excipients including buffers, which are well known in the art. The present invention can be used alone or in combination with other methods of treatment.

The anti-NGF antibodies as described herein are administered to a subject via systemic administration (e.g., intravenous or subcutaneous administration). Preferably the antibodies are administered via subcutaneous injection.

Various formulations of an anti-NGF antibody may be used for administration. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some embodiments, these agents are formulated for administration by injection (e.g., intraperitoneally, intravenously, subcutaneously, intramuscularly, intraarticularly, epidurally, intrathecally, injection into the intervertebral disc, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

In some embodiments the anti-NGF antibody, such as tanezumab, is administered in a formulation described in WO2010/032220, herein incorporated by reference.

In some embodiments, the formulation is a liquid formulation and comprises an anti-NGF antibody at a concentration of about 2.5 mg/ml, 5 mg/ml, 10 mg/ml or 20 mg/ml; and a histidine buffer.

In some embodiments, the formulation further comprises a surfactant which may be polysorbate 20. In some embodiments, the formulation further comprises trehalose dehydrate or sucrose. In some embodiments, the formulation further comprises a chelating agent, which may be EDTA; in some embodiments disodium EDTA. In some embodiments, the formulation is of pH 6.0±0.3.

In some embodiments, the formulation comprises about 2.5 mg/ml, 5 mg/ml, 10 mg/ml or 20 mg/ml tanezumab; about 10 mM histidine buffer; about 84 mg/ml trehalose dehydrate; about 0.1 mg/ml Polysorbate 20; about 0.05 mg/ml disodium EDTA; wherein the formulation is of a pH 6.0±0.3.

In some embodiments the formulation comprises about 5 mg/ml or 10 mg/ml. In some embodiments, the formulation has a total volume of about 1 ml.

In some embodiments the formulation is contained in a glass or plastic vial or syringe. In some embodiments the formulation is contained in a pre-filled glass or plastic vial or syringe.

The anti-NGF antibody can be administered every eight weeks. For repeated administrations over several doses, the treatment is sustained until a desired suppression of signs and symptoms of osteoarthritis occurs. The progress of this therapy can be monitored by conventional techniques and assays.

The dosing regimen (including the specific anti-NGF antibodies used) can vary over time. For example, in some embodiments, the dosage is 10 mg administered every eight weeks. In some embodiments the dosage is 5 mg administered every eight weeks. In some embodiments the dosage of 5 mg can be increased to 10 mg for subsequent administrations. For example, the dosage of 5 mg can be administered at start of therapy and then a dosage of 10 mg can be administered at eight weeks, with a dosage of 10 mg being administered at sixteen weeks and each subsequent eight weekly dosage. In addition, as another example, the dosage of 5 mg can be administered at start of therapy and at eight weeks, with a dosage of 10 mg being administered at sixteen weeks and each subsequent eight weekly dosage. In addition, as another example, the 5 mg dosage can be administered at start of therapy and then for one, two, or more eight weekly dosages before subsequent dosages of 10 mg every eight weeks are administered.

In some aspects in which the antibody is fasinumab (see, for example, US 2009/0041717, herein incorporated by reference), the antibody is administered at a dose of between 0.5 mg to 50 mg. In some embodiments the antibody is administered at dose between 0.5 mg and 12 mg. In some embodiments the antibody is administered at a dose of 1 mg, 2 mg, 3 mg, 4 mg, 5 mg, 6 mg, 7 mg, 8 mg, 9 mg or 10 mg. In some embodiments the antibody is administered subcutaneously or intravenously. In some embodiments the antibody is administered every four weeks or every eight weeks.

In some aspects in which the antibody is comprises the same or substantially the same amino acid sequence as fasinumab (see, for example, US 2009/0041717, herein incorporated by reference), the antibody is administered at a dose of between 0.5 mg to 50 mg. In some embodiments the antibody is administered at dose between 0.5 mg and 12 mg. In some embodiments the antibody is administered at a dose of 1 mg, 2 mg, 3 mg, 4 mg, 5 mg, 6 mg, 7 mg, 8 mg, 9 mg or 10 mg. In some embodiments the antibody administered subcutaneously or intravenously. In some embodiments the antibody is administered every four weeks or every eight weeks.

In some embodiments a loading dose (or induction dose) is administered followed by the administration of maintenance doses at a lower amount or at lower frequency.

For the purpose of the present invention, the appropriate dosage of an anti-NGF antibody will depend on the antibody employed, the type and severity of symptoms to be treated, whether the agent is administered for preventive or therapeutic purposes, previous therapy, the patient's clinical history and response to the agent, the patient's clearance rate for the administered agent, and the discretion of the attending physician. Typically the clinician will administer an anti-NGF antibody until a dosage is reached that achieves the desired result. Dose and/or frequency can vary over course of treatment. Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of symptoms.

In one embodiment, dosages for an anti-NGF antibody may be determined empirically in individuals who have been given one or more administration(s) of an anti-NGF antibody. For example, individuals are given incremental dosages of an anti-NGF antibody. To assess efficacy, an indicator of the chronic low back pain can be followed.

Administration of an anti-NGF antibody as described herein in accordance with the method in the present invention can be continuous or intermittent, depending, for example, upon the recipient's physiological condition, whether the purpose of the administration is therapeutic or prophylactic, and other factors known to skilled practitioners. The administration of an anti-NGF antibody may be essentially continuous over a preselected period of time or may be in a series of spaced doses.

In some embodiments, more than one anti-NGF antibody may be present. At least one, at least two, at least three, at least four, at least five different, or more anti-NGF antibodies can be present. Generally, those anti-NGF antibodies may have complementary activities that do not adversely affect each other.

In some embodiments, the anti-NGF antibody may be administered in combination with the administration of one or more additional therapeutic agents.

In some embodiments, an anti-NGF antibody administration is combined with a treatment regimen further comprising a traditional therapy including surgery.

### Formulations

Therapeutic formulations of the anti-NGF antibody used in accordance with the present invention are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the anti-NGF antibody are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic anti-NGF antibody compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an anti-NGF antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

In embodiments that refer to a method of treating chronic low back pain (CLBP) as described herein, such embodiments are also further embodiments of an anti-NGF antibody for use in that treatment, or alternatively of the use of an anti-NGF antibody in the manufacture of a medicament for use in that treatment.

### Kits

The invention also provides kits comprising any or all of the anti-NGF antibodies described herein. Kits of the invention include one or more containers comprising an anti-NGF antibody described herein and instructions for use in accordance with any of the methods of the invention described herein. Generally, these instructions comprise a description of administration of the anti-NGF antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an anti-NGF antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-NGF antibody. The container may further comprise a second pharmaceutically active agent.

Kits may optionally provide additional components such as buffers and interpretive information. Normally, the kit comprises a container and a label or package insert(s) on or associated with the container.

### Example 1

Study Design

This study (termed “Study 1059”) was a randomized, double-blind, placebo- and active-controlled, multicenter, parallel-group Phase 3 study of the efficacy and safety of tanezumab when administered by SC injection for up to 56 weeks in adult patients with chronic low back pain. Patients had low back pain at baseline with the primary location between the 12th thoracic vertebra and the lower gluteal folds, classified as Category 1 or 2 according to the classification of the Quebec Task Force in Spinal Disorders, a duration of chronic low back pain of ≥3 months, moderate to severe chronic low back pain as demonstrated by an average LBPI score >5 over at least 4 daily assessments during the 5 days prior to the day of randomization, and a baseline Patient Global Assessment of Low Back Pain score of “fair”, “poor” or “very poor”. Patients were also required to have documented history of previous inadequate treatment response to at least 3 different categories of agents commonly used and generally considered effective for the treatment of chronic low back pain. Patients were required to discontinue all medications for the treatment of chronic low back pain during the primary efficacy assessment period (up to Week 16). Patients with a diagnosis of osteoarthritis of the knee or hip as defined by the American College of Rheumatology combined clinical and radiographic criteria or who had Kellgren Lawrence Grade ≥2 radiographic evidence of hip osteoarthritis or Kellgren Lawrence Grade ≥3 radiographic evidence of knee osteoarthritis were excluded.

Approximately 1800 patients were planned to be randomized initially in a 2:2:2:3 ratio to 1 of 4 following treatment groups:

1. Placebo administered SC at an 8-week interval plus placebo matching tramadol PR up to Week 16. At the Week 16 visit, patients in this group were switched in a blinded fashion in a 1:1 ratio to either tanezumab 5 mg or tanezumab 10 mg administered SC at an 8 week interval plus placebo matching tramadol PR to Week 56;

2. Tanezumab 5 mg SC administered at an 8-week interval plus placebo matching tramadol PR to Week 56;

3. Tanezumab 10 mg SC administered at an 8-week interval plus placebo matching tramadol PR to Week 56;

4. Oral tramadol PR 100-300 mg daily plus placebo administered SC at an 8-week interval to Week 56.

To achieve the initial randomization and intended re-randomization at Week 16 for placebo patients, a randomization ratio 1:1:2:2:3 for placebo→tanezumab 5 mg (at Week 16), placebo→tanezumab 10 mg (at Week 16), tanezumab 5 mg, tanezumab 10 mg, and tramadol PR was used at the beginning of the trial.

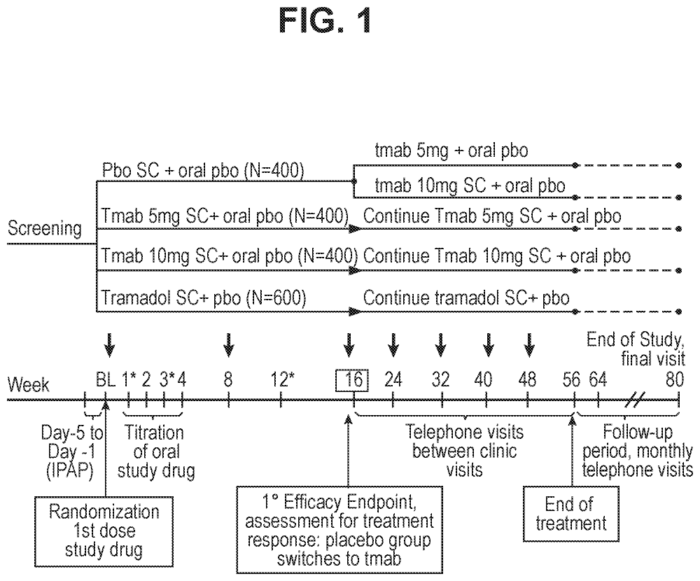

The study was designed with a total duration (post randomization) of up to 80 weeks and consisted of three periods: (1) a Screening Period, (2) a Double-blind Treatment Period (comprised of a 16-week Primary Efficacy Phase and a 40-week Long-Term Safety and Efficacy Phase), and (3) a 24-week Follow-up Period (FIG. 1).

Patient Population

Patients were required to have low back pain with the primary location between the 12th thoracic vertebra and the lower gluteal folds, classified as Category (pain without radiation) or 2 (pain with proximal radiation [above the knee]) according to the classification of the Quebec Task Force in Spinal Disorders and a duration of chronic low back pain of ≥3 months. Table 2 summarizes the requirements for analgesic medication usage prior to Screening and the requirements for LBPI and Patient's Global Assessment (PGA) of CLBP. The current study enrolled patients who were more refractory to standard of care treatments for CLBP and had higher LBPI scores at Baseline.

TABLE 2
Summary of Key Entry Criteria

| Entry Criteria | Study 1059 (SC) |
|---|---|
| Requirements for | Documented history of previous inadequate |
| medication usage | treatment response to at least 3 different |
| prior to | categories of agents commonly used and generally |
| Screening | considered effective for the treatment of CLBP: |
| | acetaminophen/low-dose NSAIDs |
| | prescription NSAIDs |
| | opioids (not tramadol) |
| | tapentadol, |
| | tricyclic antidepressants |
| | benzodiazepines or skeletal muscle relaxants |
| | lidocaine patch |
| | duloxetine or other serotonin-norepinephrine |
| | reuptake inhibitors |
| LBPI at Baseline | ≥5 |
| PGA at Baseline | Fair, poor or very poor |

CLBP = chronic low back pain;
LBPI = low back pain intensity;
NSAIDs = nonsteroidal anti-inflammatory drugs
PGA = Patient's Global Assessment;
RMDQ = Roland Morris Disability Questionnaire;
SC = subcutaneous

Table 3 Key Demographic and Baseline Characteristics—Safety Population

The baseline characteristics of the patients' CLBP across treatment groups are summarized for the current study in Table 3. The range of mean scores across treatment groups for LBPI and RMDQ suggest the patients enrolled in this study (LBPI =7.17 to 7.24; RMDQ=14.81 to 15.10) had more severe CLBP than the patients enrolled in prior studies. This study had approximately 15% fewer patients with degenerative joint disease/osteoarthritis as assessed by the principal investigator compared to prior study and about 5% more patients with degenerative disc disease. Based on assessments from the painDETECT screening tool to predict the likelihood of a neuropathic pain component being present in individual patients, 68% of the patients enrolled in this study had a predominant non-neuropathic pain component and approximately 13% likely had a neuropathic pain component.

TABLE 3
Summary of Key Baseline Characteristics

| Baseline Characteristic (range of | Study (SC) |
|---|---|
| mean scores across treatment groups) | (N = 1825) |
| LBPI^{a} | 7.17-7.24 |
| RMDQ^{b} | 14.81-15.10 |
| PGA of CLBP^{c} | 3.47-3.53 |

Primary etiology assessment,^{d} n (%)

| | | |
|---|---|---|
| Degenerative disc disease | 550 | (30.14) |
| Degenerative joint disease/OA | 443 | (24.27) |
| Injury/muscular strain | 591 | (32.38) |
| Injury/muscular strain: degenerative | 1 | (0.05) |
| disc disease, herniated disc | | |
| Other | 240 | (13.15) |

painDETECT Category,^{e} n (%)

| | | |
|---|---|---|
| ≤12 (neuropathic component | 1249 | (68.44) |
| unlikely) | | |
| 13 to 18 | 344 | (18.85) |
| ≥19 (neuropathic component likely) | 230 | (12.60) |

CLBP = chronic low back pain;
LBPI = low back pain intensity;
OA = osteoarthritis;
PGA = Patient's Global Assessment;
RMDQ = Roland Morris Disability Questionnaire;
SC = subcutaneous
^{a}Assessed with an 11-point numeric rating scale ranging from 0 (no pain) to 10 (worst possible pain)
^{b}CLBP-specific assessment of physical function with scores ranging from 0 to 24 (lower scores indicate better function).
^{c}Global evaluation that utilizes 5-point Likert scale with a score of 1 being best (very good) and a score of 5 being worst (very poor).
^{d}Principal Investigators' assessment of the primary etiology of the patients' CLBP based on patient report, history and physical examination, medical records or report from patient's physician, or imaging report.
^{e}Tool to screen for the prevalence of neuropathic pain components in CLBP patients, with scores ≤12 indicating that a neuropathic component is unlikely and scores ≥19 indicating a neuropathic component is likely. For scores of 13-19, the result is uncertain, i.e. a neuropathic pain component can be present. Range of scores −1 to 38.

Table 4 summarizes the data for the primary endpoint (LBPI change from Baseline to Week 16) and key secondary endpoint (RMDQ change from Baseline to Week 16) included in this study. Across 3 out of 4 efficacy endpoints (LBPI and RMDQ change from Baseline to Week 16 and 250% improvement in LBPI); the placebo response is larger in this study than a prior study. The treatment differences for tanezumab 10 mg vs. placebo for the LBPI endpoints were more modest in the current study compared to the prior study. Nevertheless, all of the treatment comparisons between tanezumab 10 mg and placebo for LBPI were statistically significant in both studies. For RMDQ (change from Baseline to Week 16), the treatment response with bath dose strengths of tanezumab were larger in the current study compared to the prior study.

The active comparator in this study was tramadol PR whereas the active comparator in a prior study was naproxen 500 mg BID. Tramadol PR was not significantly different from placebo for any of the endpoints summarized in Table 4. However, naproxen 500 mg BID demonstrated significant differences relative to placebo for the LPBI endpoints, but not the RMDQ endpoint. A systematic review and meta-analysis of opioid analgesics for the treatment of low back pain published in JAMA Intern Med (2016; 176(7):958-968) indicated studies of tramadol have had mixed efficacy results for improvements in pain with some studies differentiating from placebo and some not doing so. The treatment duration of these studies ranged from 4 to 12 weeks

TABLE 4
Summary of LBPI, RMDQ, and Proportion of Patients
with ≥50% Improvement in LBPI at Week 16

| | Plc | Tan 5 mg | Tan 10 mg | Tramadol PR |
|---|---|---|---|---|
| N | 406 | 407 | 407 | 605 |

Primary Endpoint: LBPI^{a} Change from Baseline to Week 16

| | | | | | | | | |
|---|---|---|---|---|---|---|---|---|
| LS Mean | −2.68 | (0.15) | −2.98 | (0.14) | −3.08 | (0.14) | −2.81 | (0.12) |
| (SE) | | | | | | | | |

| | | | | | | |
|---|---|---|---|---|---|---|
| Difference | −0.30 | (0.19) | −0.40 | (0.18) | −0.12 | (0.17) |
| vs. plc | | | | | | |
| (SE) | | | | | | |

| | | | |
|---|---|---|---|
| p-value | 0.112 | 0.028* | 0.462 |

Key Secondary Endpoint: RMDQ^{b} Change from Baseline to Week 16

| | | | | | | | | |
|---|---|---|---|---|---|---|---|---|
| LS Mean | −4.95 | (0.36) | −6.27 | (0.35) | −6.69 | (0.35) | −5.21 | (0.30) |
| (SE) | | | | | | | | |

| | | | | | | |
|---|---|---|---|---|---|---|
| Difference | −1.32 | (0.45) | −1.74 | (0.46) | −0.26 | (0.42) |
| vs. plc | | | | | | |
| (SE) | | | | | | |

| | | | |
|---|---|---|---|
| p-value | 0.004 | <0.001* | 0.541 |

Secondary Endpoint: Proportion of Patients with ≥50%
Improvement in LBPI at Week 16

| | | | | | | | | |
|---|---|---|---|---|---|---|---|---|
| n (%) | 151 | (37.4) | 176 | (43.3) | 188 | (46.3) | 259 | (42.8) |

| | | | | | | |
|---|---|---|---|---|---|---|
| OR vs. plc | 1.28 | (0.97, 1.70) | 1.45 | (1.09, 1.91) | 1.25 | (0.97, 1.62) |
| (95% CI) | | | | | | |

| | | | |
|---|---|---|---|
| p-value | 0.0846 | 0.010* | 0.0848 |

Secondary Endpoint: LBPI Change from Baseline to Week 2 in LBPI

| | | | | | | | | |
|---|---|---|---|---|---|---|---|---|
| LS Mean | −1.17 | (0.09) | −1.54 | (0.09) | −1.59 | (0.09) | 1.36 | (0.08) |
| (SE) | | | | | | | | |

| | | | | | | |
|---|---|---|---|---|---|---|
| Difference | −0.37 | (0.12) | −0.42 | (0.12) | −0.19 | (0.11) |
| vs. plc | | | | | | |
| (SE) | | | | | | |

| | | | |
|---|---|---|---|
| p-value | 0.002 | <0.001* | 0.077 |

LBPI = low back pain intensity;
LS = least squares;
OR = odds ratio;
plc = placebo;
PR = prolonged release;
RMDQ = Roland Morris Disability Questionnaire;
SC = subcutaneous;
SE = standard error;
tan = tanezumab
^{a}Assessed with an 11-point numeric rating scale ranging from 0 (no pain) to 10 (worst possible pain)
^{b}CLBP-specific assessment of physical function with scores ranging from 0 to 24 (lower scores indicate better function).
*Statistically significant per the pre-specified testing procedure

Improvements in LBPI and RMDQ, relative to baseline and tramadol, were maintained throughout the study but were not significantly better than tramadol (N=605; mean dose=209 mg/day) at week 56 for tanezumab 5 mg (N=407; LS mean [95% CI] difference=−0.11 [−0.51,0.28] for LBPI and −0.44 [−1.47,0.58] for RMDQ) or 10 mg (N=407; LS mean [95%] difference=−0.21 [−0.61,0.18] for LBPI and −0.83 [−1.84,0.18] for RMDQ).

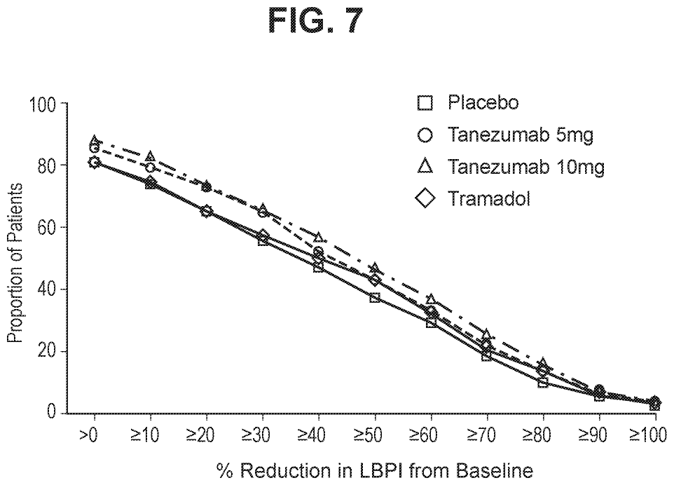

The percentage of patients achieving ≥30% improvement in LBPI at week 16 was greater in the tanezumab 5 mg (64.8%) and tanezumab 10 mg (65.5%) groups compared to placebo (55.9%). Odds ratio (95% CI) versus placebo was 1.45 (1.09, 1.92; p=0.0101) for tanezumab 5 mg and 1.50 (1.13, 1.99; p=0.0054) for tanezumab 10 mg. As shown in FIG. 7, the proportion of patients with a >0% to ≥90% improvement in LBPI at week 16 was larger in both tanezumab groups than in the placebo group, though the treatment difference incrementally reduced with increasing level of response threshold. In addition, the percentage of patients achieving ≥30% improvement in LBPI at week 16 was not different between the placebo (55.9%) and tramadol (57.9%) groups; odds ratio (95% CI) versus placebo was 1.08 (0.84, 1.39; p=0.5493). The percentage of patients achieving 230% improvement in LBPI at week 16 was greater in the tanezumab 5 mg (64.8%) and tanezumab 10 mg (65.5%) groups compared to tramadol (57.9%). Odds ratio (95% CI) versus tramadol was 1.34 (1.03, 1.74; p=0.0269) for tanezumab 5 mg and 1.38 (1.07, 1.80; p=0.0144) for tanezumab 10 mg. At week 56, LS mean (95% CI) difference, versus tramadol, in LBPI was −0.21 (−0.61, 0.18; P=0.2887) for tanezumab 10 mg and −0.11 (−0.51, 0.28; P=0.5763) for tanezumab 5 mg.

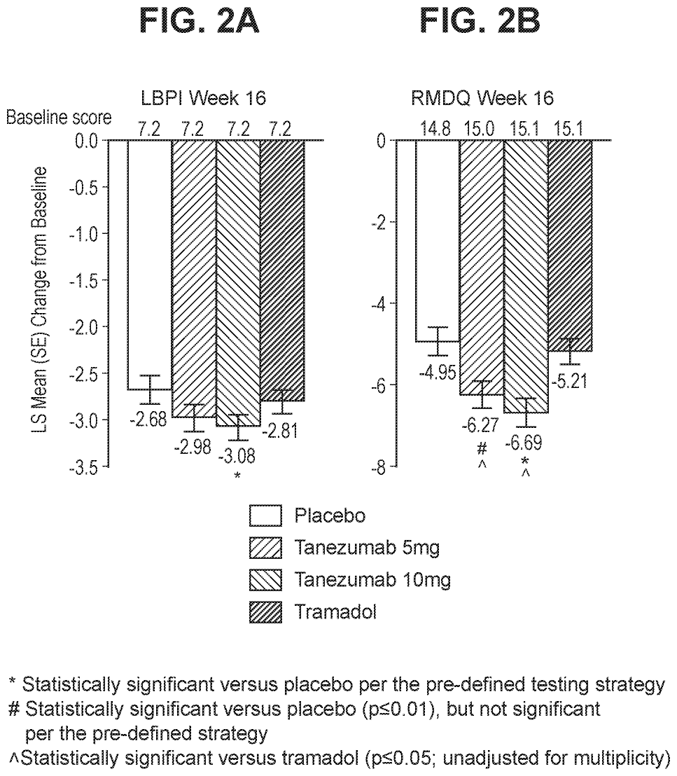

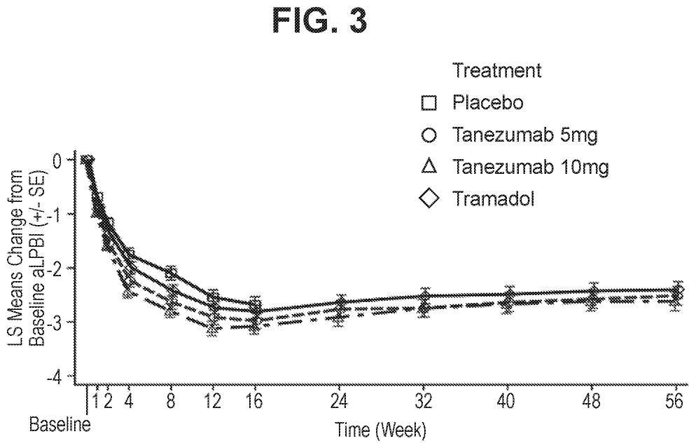

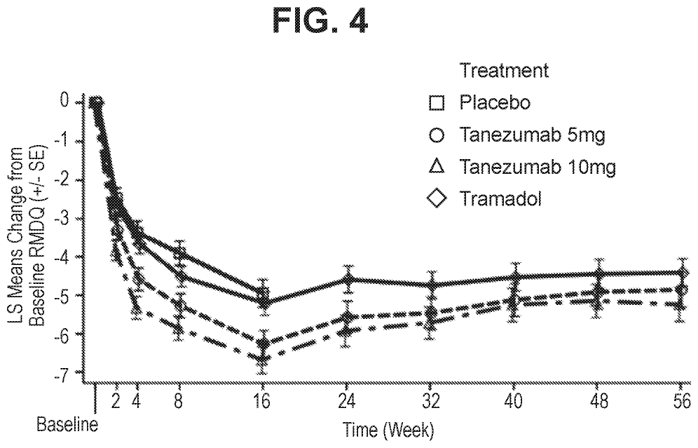

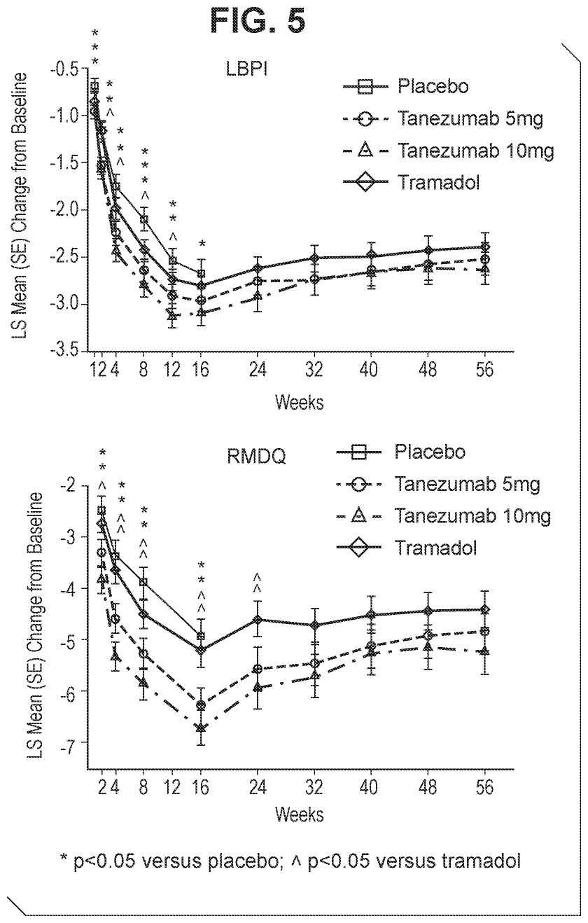

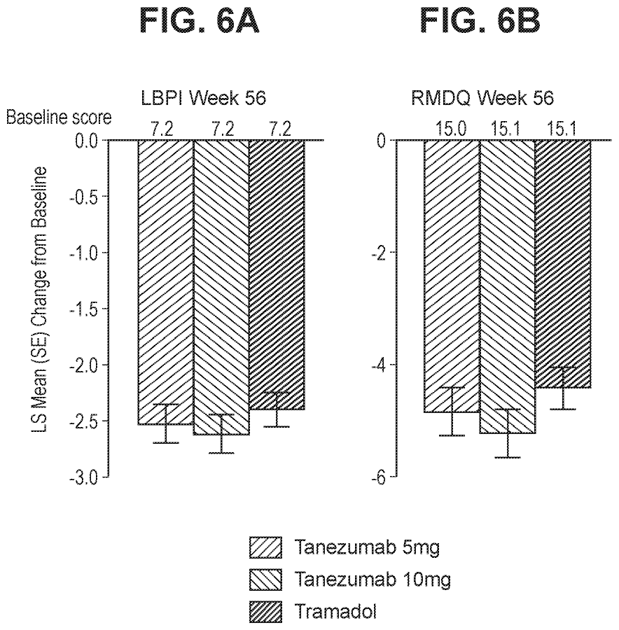

FIG. 2 shows the change from baseline for LBPI and RMDQ scores from baseline at week 16. FIG. 3 shows the change from baseline for LBPI score up to week 56 (ITT population, multiple imputation). FIG. 4 shows the change from baseline for RMDQ up to week 56 (ITT population). FIG. 5 shows the change in both LPBI and RMDQ scores throughout the 56 week treatment period. FIG. 6 shows the change from baseline for LBPI and RMDQ scores at week 56. FIG. 7 shows the proportion of patients with a >0% to ≥90% improvement in LBPI at week 16.

Safety

The overall adverse event profile with tanezumab treatment observed in this study was generally consistent with earlier studies conducted in patients with CLBP. In this study, the overall incidence of adverse events during the treatment period in either tanezumab treatment group was lower than in the tramadol PR treatment group both up to Week 16 when the primary endpoint was assessed and during the 56-week treatment period. The incidence of serious adverse events during the 56-week treatment period was highest in the tanezumab 10 mg treatment group, followed by the tramadol PR treatment group and the tanezumab 5 mg treatment. The highest incidence of discontinuations from treatment due to an adverse event up to Week 16 and during the 56-week treatment period occurred in the tramadol PR treatment group relative to the tanezumab 10 mg and 5 mg treatment groups.

In this study, patients with a diagnosis of osteoarthritis of the knee or hip as defined by the American College of Rheumatology (ACR) combined clinical and radiographic criteria or who had Kellgren Lawrence Grade ≥2 radiographic evidence of hip osteoarthritis (OA; definite osteophytes, possible joint space narrowing) or Kellgren Lawrence Grade ≥3 radiographic evidence of knee OA (moderate osteophytes, definite joint space narrowing, some sclerosis, possible bone-end deformity) were excluded. Therefore patients who had Kellgren Lawrence Grade ≤2 radiographic evidence of knee OA but who did not meet ACR criteria and did not have pain associated with their knee OA were eligible to participate in the study.

Of the 30 total patients (1.6% of total patients) who had a joint safety event during the 80-week observation period that met the criteria for adjudication, there was a higher number of tanezumab-treated patients (26/1008=2.6% of patients) requiring adjudication compared to tramadol PR-treated patients (4/602=0.7%). No patients in the placebo treatment group had joint safety events that required adjudication; therefore there were no adjudicated joint safety endpoints in the placebo treatment group.

The incidence of the composite joint safety endpoint (rapidly progressive OA [RPOA], primary osteonecrosis, subchondral insufficiency fracture, pathologic fracture) was highest in the tanezumab 10 mg treatment group (2.6%) compared to the tanezumab 5 mg treatment group (1.0%) and the tramadol PR treatment group (0.2%). Across the treatment groups, the knee was the affected joint in 16/19 patients (84.2%) who had an adjudicated event included in the composite joint safety endpoint. The baseline Kellgren Lawrence Grades for the affected joints were 7 patients with Grade 0 (no joint space narrowing or reactive changes), 6 patients with Grade 1 (doubtful joint space narrowing, possible osteophytic lipping), and 5 patients with Grade 2 radiographic evidence of OA. Kellgren Lawrence Grade was not evaluated on shoulder x-rays, but the patient who had an endpoint in the shoulder did not have radiographic evidence of osteophytes on the Screening shoulder x-rays. Additional evaluation of the baseline characteristics and medical history for these patients will be performed to determine if any predisposing risk factors can be identified.

Approximately 1.4% of tanezumab-treated patients (14/1008) had an adjudicated event of RPOA compared to 0.2% of tramadol PR-treated patients (1/602). In total 13 patients had a joint safety event adjudicated to RPOA Type 1 (7 in the tanezumab 10 mg treatment group [1.4%], 5 in the tanezumab 5 mg treatment group [1.0%], and 1 in the tramadol PR treatment group [0.2%]). Two patients, both in the tanezumab 10 mg treatment group (0.4%), had joint safety events adjudicated to RPOA Type 2. The ratio of RPOA Type 1 to RPOA Type 2 for tanezumab was 6:1. Four patients in the tanezumab 10 mg treatment group (0.8%) had joint safety events adjudicated to subchondral insufficiency fracture. Two patients had joint safety events adjudicated to normal progression of OA (1 patient in the tanezumab 5 mg treatment group and 1 patient in the tanezumab 10 mg treatment group).

Among the 30 total patients who had a joint safety event meeting the criteria for adjudication, a total of 7 patients had a single total joint replacement (TJR) during the study observation period (Baseline to Week 80 or 26 weeks after the Treatment Period, whichever was later). All of the patients were treated with tanezumab 10 mg. The joints replaced were the knee (n=4), hip (n=1) and shoulder (n=2). In addition, all TJRs were associated with an adverse event and/or adjudicated to a composite joint safety event (i.e., the surgery was not considered elective). Two of these patients had an adjudication outcome of RPOA Type 1, 2 patients had an adjudication outcome of RPOA Type 2, and 1 patient had an adjudication outcome of subchondral insufficiency fracture. The remaining 2 patients who had a TJR had adjudication outcomes of Other (meniscal tear and trauma).

Interpretation of Primary Results

The primary objective of the study was achieved with tanezumab 10 mg, but not with tanezumab 5 mg. There was statistically significant improvement in the primary efficacy endpoint, change from baseline to Week 16 in the LBPI score, for the tanezumab 10 mg treatment versus placebo treatment. No statistically significant improvement was demonstrated with tanezumab 5 mg treatment versus placebo treatment at Week 16.

Tramadol PR only modestly improved the LBPI compared to placebo at Week 16 and the treatment difference was not statistically significant. The changes from baseline to Week 16 in the LBPI with both the tanezumab 10 mg and tanezumab 5 mg treatment were favorable compared to tramadol PR although neither treatment difference reached statistical significance.

Treatment with tanezumab 10 mg provided superior responder rate (250% improvement in the LBPI at Week 16) and superior improvement in physical function (change from baseline at Week 16 in the RMDQ) compared to placebo treatment, and demonstrated an onset of effect at Week 2.

Tanezumab 5 mg treatment provided a larger improvement in RMDQ versus placebo treatment at Week 16, but it is not possible to draw a conclusion of superiority of tanezumab 5 mg for this comparison due to the lack of a significant treatment difference of tanezumab 5 mg versus placebo in the LBPI per the pre-specified testing procedure. Both tanezumab treatments showed significantly larger improvements in RMDQ at Week 16 than tramadol PR treatment at α=0.05 (with no multiplicity correction).

The treatment differences for change from baseline to Week 56 for LBPI and RMDQ were only modestly larger in both the tanezumab 10 mg and 5 mg treatment groups relative to the tramadol PR treatment group and the differences were not statistically significant. Improvement in pain and function was maintained long-term.

The adverse event profile with tanezumab treatment observed in this study was consistent with earlier studies conducted in patients with CLBP. The overall incidence of adverse events during the treatment period in either tanezumab treatment group was lower than in the tramadol treatment group both up to Week 16 when the primary endpoint was assessed and during the 56-week treatment period. The incidence of serious adverse events during the 56-week treatment period was highest in the tanezumab 10 mg treatment group, followed by the tramadol PR treatment group and the tanezumab 5 mg treatment. The highest incidence of discontinuations from treatment due to an adverse event up to Week 16 and during the 56-week treatment period occurred in the tramadol PR treatment group relative to the tanezumab 10 mg and 5 mg treatment groups.

The incidence and observation-time adjusted rates of composite joint safety endpoint (rapidly progressive OA, primary osteonecrosis, subchondral insufficiency fracture, pathologic fracture) were highest in the tanezumab 10 mg treatment group (2.6% and 25.7 events/1000 patient-years) compared to the tanezumab 5 mg treatment group (1.0% and 10.0 events/1000 patient-years) and the tramadol treatment group (0.2% and 1.9 events/1000 patient-years). No composite joint safety endpoints were observed in the placebo treatment group. A total of 13 patients had joint safety events that were adjudicated to rapidly progressive OA type 1 (tanezumab 5 mg-5 patients, tanezumab 10 mg-7 patients, and tramadol-1 patient). Two patients in the tanezumab 10 mg treatment group had adjudicated events of rapidly progressive OA type 2, and four patients, also in the tanezumab 10 mg treatment group, had adjudicated events of subchondral insufficiency fracture.

Among the 30 patients who had events adjudicated for joint safety outcomes, 7 patients in the tanezumab 10 mg treatment group underwent total joint replacement during the study observation period.

The study demonstrates the potential of anti-NGF antibodies, including tanezumab, to treat individuals suffering from moderate-to-severe chronic low back pain who have been unable to achieve relief with currently available medicines. Such patients living with chronic low back pain suffer from constant pain, which significantly impacts their ability to perform everyday tasks. The use of anti-NGF antibodies, including tanezumab, represents an innovative non-opioid treatment to help address this life-altering and debilitating condition.

### Example 2

### Study Design

This second study (termed “Study 1063) was a randomized, double-blind, active-controlled, multicenter, parallel-group Phase 3 study in Japan to evaluate the safety and efficacy of tanezumab when administered by SC injection for up to 56 weeks in subjects with chronic low back pain (CLBP). Patients had low back pain at baseline with the primary location between the 12th thoracic vertebra and the lower gluteal folds, classified as Category 1 or 2 according to the classification of the Quebec Task Force in Spinal Disorders, a duration of CLBP of ≥3 months, moderate to severe CLBP as demonstrated by an average Low Back Pain Intensity (LBPI) score of ≥5 over at least 4 daily assessments during the 5 days prior to the day of randomization, and a baseline Patient's Global Assessment of Low Back Pain of “fair”, “poor” or “very poor”. Patients were also required to be experiencing some benefit from their current stable dose regimen of oral therapy of NSAID (celecoxib 200 mg/day [100 mg BID], loxoprofen 120 to 180 mg/day or meloxicam 5 to 15 mg/day) treatment, but still required additional pain relief at screening. Patients were required to discontinue all medications (except for muscle relaxants, pregabalin, gabapentin and anti-depressants which had been taken with stable dose since at least 30 days prior to screening) for the treatment of CLBP until week 16. Patients with a diagnosis of osteoarthritis of the knee or hip as defined by the American College of Rheumatology combined clinical and radiographic criteria or who had Kellgren Lawrence Grade ≥2 radiographic evidence of hip osteoarthritis or Kellgren Lawrence Grade ≤3 radiographic evidence of knee osteoarthritis were excluded.

Approximately 200 patients (170-220 patients, approximately 66 patients [56-73 patients] per treatment group) were planned for randomization to one of 3 treatment groups in a 1:1:1 ratio in terms of study feasibility and safety evaluation. However, it was acceptable to randomize more than 220 patients from the safety perspective.

Patients received a total of seven SC injections, separated by 8 weeks (tanezumab or

placebo), and daily celecoxib 100 mg BID through Week 56. Treatment groups were as follows:
1. Tanezumab 5 mg SC and placebo for celecoxib BID;
2. Tanezumab 10 mg SC and placebo for celecoxib BID;
3. Placebo for tanezumab SC and celecoxib BID.

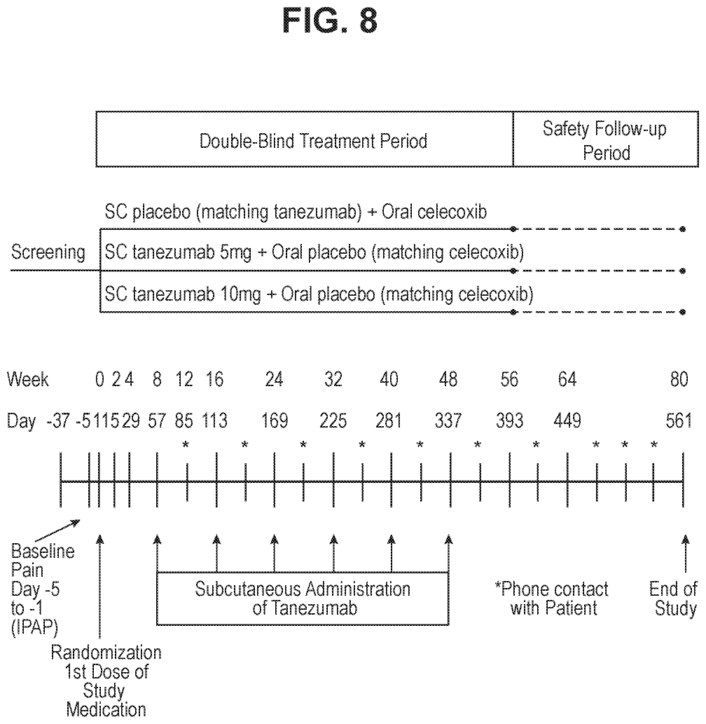

This study was designed with a total (post-randomization) duration of 80 weeks and consisted of three periods: (1) a Screening period (up to a maximum of 37 days), (2) a Double-blind Treatment period (56 weeks), and (3) a Safety Follow-up (24 weeks) Period (FIG. 8). The Screening Period included a Washout Period (lasting 2-32 days) if required, and an Initial Pain Assessment Period (IPAP) (5 days prior to Randomization/Baseline; minimum 4 days).

At the Week 16 visit, patients must have had a 30% or greater reduction in average LBPI score relative to Baseline and a 15% or greater reduction in the average LBPI score from Baseline at any week from Week 1 to Week 15. Patients who did not meet these response criteria were discontinued from the Treatment Period and entered the Early Termination Safety Follow-up Period.

### Patient Population

The Intent-to-Treat (ITT) analysis set included all patients who were randomized and received at least one dose of SC study medication (either tanezumab or placebo). This analysis set was primary for all efficacy endpoints, which were analyzed according to randomization assignment, and was labeled as the ‘ITT population’.

The Safety analysis set included all patients who received at least one dose of SC study treatment. This analysis set was primary for all safety endpoints, which were analyzed according to treatment received, and was labeled as the ‘Safety population’. In this study, the ITT and Safety analysis sets were identical.

A total of 277 patients were randomized; 92 patients were randomized to tanezumab 5 mg, 93 to tanezumab 10 mg, and 92 to celecoxib. Further patient disposition is shown in Table 5 and Table 6.

TABLE 5
Patient Disposition

| | tanezumab | | tanezumab | | | |
|---|---|---|---|---|---|---|
| | 5 mg | | 10 mg | | celecoxib | |
| Randomized | 92 | | 93 | | 92 | |
| Not Treated | 0 | | 0 | | 0 | |
| Safety Population, n (%) | 92 | (100.0) | 93 | (100.0) | 92 | (100.0) |
| ITT Population, n (%) | 92 | (100.0) | 93 | (100.0) | 92 | (100.0) |
| Completed Treatment Phase^{a}, n (%) | 62 | (67.4) | 43 | (46.2) | 43 | (46.7) |
| Discontinued Treatment Phase^{a}, n (%) | 30 | (32.6) | 50 | (53.8) | 49 | (53.3) |
| Adverse Event | 3 | (3.3) | 5 | (5.4) | 4 | (4.3) |
| Lost to Follow-Up | 0 | | 1 | (1.1) | 0 | |
| Withdrawal By Subject | 0 | | 1 | (1.1) | 4 | (4.3) |
| Insufficient Clinical Response | 1 | (1.1) | 4 | (4.3) | 1 | (1.1) |
| Other | 1 | (1.1) | 4 | (4.3) | 5 | (5.4) |
| Patient Meets Protocol Specified | 25 | (27.2) | 35 | (37.6) | 35 | (38.0) |
| Pain Criteria for Discontinuation | | | | | | |
| Completed Study^{ab}, n (%) | 88 | (95.7) | 82 | (88.2) | 87 | (94.6) |
| Discontinued Study^{a}, n (%) | 4 | (4.3) | 11 | (11.8) | 5 | (5.4) |
| Adverse Event | 1 | (1.1) | 1 | (1.1) | 0 | |
| Lost to Follow-Up | 1 | (1.1) | 1 | (1.1) | 0 | |
| Withdrawal By Subject | 1 | (1.1) | 3 | (3.2) | 3 | (3.3) |
| Insufficient Clinical Response | 0 | | 2 | (2.2) | 0 | |
| Other | 1 | (1.1) | 4 | (4.3) | 2 | (2.2) |

^{a}Denominator is number of subjects in the Safety Population.
^{b}Patients completed the study if they completed the safety follow-up period, regardless of whether they completed the treatment phase.

TABLE 6
Patient Disposition for Safety Follow-Up

| | tanezumab | | tanezumab | | | |
|---|---|---|---|---|---|---|
| | 5 mg | | 10 mg | | celecoxib | |
| Safety Population | 92 | | 93 | | 92 | |
| Completed Treatment Phase | 62 | (67.4) | 43 | (46.2) | 43 | (46.7) |
| Completed Safety Follow-Up | 61 | (66.3) | 42 | (45.2) | 43 | (46.7) |
| Discontinued Safety | 1 | (1.1) | 1 | (1.1) | 0 | |
| Follow-Up | | | | | | |
| Did not enter Safety | 0 | | 0 | | 0 | |
| Follow-Up | | | | | | |
| Discontinued Treatment | 30 | (32.6) | 50 | (53.8) | 49 | (53.3) |
| Phase | | | | | | |
| Completed Safety Follow-Up | 27 | (29.3) | 40 | (43.0) | 44 | (47.8) |
| Discontinued Safety | 2 | (2.2) | 4 | (4.3) | 2 | (2.2) |
| Follow-Up | | | | | | |
| Did not enter Safety | 1 | (1.1) | 6 | (6.5) | 3 | (3.3) |
| Follow-Up | | | | | | |

The demographic and baseline characteristics (Table 7) were similar across the three treatment groups.

TABLE 7
Key Demographic and Baseline Characteristics

| | tanezumab | | tanezumab | | | |
|---|---|---|---|---|---|---|
| | 5 mg | | 10 mg | | celecoxib | |
| | (N = 92) | | (N = 93) | | (N = 92) | |
| Age (years) | | | | | | |
| Mean (Range) | 53.3 | (22, 79) | 52.3 | (21, 78) | 54.3 | (19, 81) |
| Sex [n(%)] | | | | | | |
| Male | 55 | (59.8) | 49 | (52.7) | 54 | (58.7) |
| Female | 37 | (40.2) | 44 | (47.3) | 38 | (41.3) |
| LBPI^{a} at Baseline | | | | | | |
| Mean (SD) | 6.74 | (0.97) | 6.82 | (1.09) | 6.72 | (1.00) |
| RMDQ^{b} at Baseline | | | | | | |
| Mean (SD) | 8.27 | (5.02) | 8.12 | (4.86) | 7.75 | (4.95) |
| Primary Etiology Assessment | | | | | | |
| [n(%)] | | | | | | |
| Degenerative Disc Disease | 32 | (34.8) | 40 | (43.0) | 38 | (41.3) |
| Degenerative Joint | 13 | (14.1) | 11 | (11.8) | 17 | (18.5) |
| Disease/OA | | | | | | |
| Injury/Muscular Strain | 6 | (6.5) | 0 | | 1 | (1.1) |
| Other | 41 | (44.6) | 42 | (45.2) | 36 | (39.1) |
| PainDetect^{c} Category [n(%)] | | | | | | |
| <=12 | 80 | (87.0) | 79 | (84.9) | 78 | (84.8) |
| 13 to 18 | 5 | (5.4) | 10 | (10.8) | 8 | (8.7) |
| >=19 | 7 | (7.6) | 4 | (4.3) | 6 | (6.5) |
| PainDetect^{d} Baseline Score | | | | | | |
| Mean (SD) | 6.79 | (5.74) | 6.71 | (5.21) | 7.36 | (5.76) |
| Quebec Task Force [n(%)] | | | | | | |
| Category 1: Pain without | 77 | (83.7) | 82 | (88.2) | 79 | (85.9) |
| radiation | | | | | | |
| Category 2: Pain plus | 15 | (16.3) | 11 | (11.8) | 13 | (14.1) |
| radiation to extremity, proximally | | | | | | |

| | | | |
|---|---|---|---|
| Category 3 or greater | 0 | 0 | 0 |

^{a}LBPI scores range from 0 (no pain) to 10 (worst possible pain).
^{b}Roland Morris Disability Questionnaire (RMDQ) total scores range from 0 to 24 with a lower score indicating better function.
^{c}PainDetect is a tool to screen for the prevalence of neuropathic pain components in CLBP patients, with scores ≤12 indicating that a neuropathic component is unlikely and scores ≥19 indicating a neuropathic component is likely.
^{d}PainDetect total score ranges from −1 to 38 with higher scores indicating higher levels of neuropathic pain.

### Safety

Table 8 summarizes treatment-emergent adverse events during the 56-week treatment period. Adverse events were reported more frequently for the celecoxib group than for the tanezumab 5 mg group, while the tanezumab 10 mg group reported the fewest adverse events. The incidence of serious adverse events during the treatment period was highest in the tanezumab 10 mg group, followed by the tanezumab 5 mg group and the celecoxib group. Few patients discontinued treatment due to an adverse event in all groups.

TABLE 8
Incidence of Treatment-Emergent Adverse Events during the
Treatment Period (all causalities) - Safety Population

| | tanezumab | tanezumab | |
|---|---|---|---|
| | 5 mg | 10 mg | celecoxib |
| | (N = 92) | (N = 93) | (N = 92) |
| Number (%) of Patients | n (%) | n (%) | n (%) |
| Adverse Event | 58 (63.0) | 51 (54.8) | 62 (67.4) |
| Serious Adverse Event | 4 (4.3) | 9 (9.7) | 2 (2.2) |
| Severe Adverse Event | 3 (3.3) | 2 (2.2) | 1 (1.1) |
| Discontinued study drug | 3 (3.3) | 5 (5.4) | 4 (4.3) |
| due to Adverse Event | | | |
| Discontinued from study | 1 (1.1) | 1 (1.1) | 0 |
| due to Adverse Event | | | |
| Discontinued study drug | 2 (2.2) | 4 (4.3) | 4 (4.3) |
| due to Adverse Event and | | | |
| continued study | | | |

The most frequent adverse events (≥3% in any treatment group) during the treatment period are shown in Table 9. Nasopharyngitis, fall, and contusion were reported more frequently in bath tanezumab groups than in the celecoxib group (>1% difference between treatment groups). Arthralgia, pyrexia, diarrhoea, gastroenteritis, and hypertension were reported more frequently in the celecoxib group than in both tanezumab groups (>1% difference between treatment groups).

TABLE 9
Incidence of most frequent (≥3%) Treatment-emergent Adverse Events
during the Treatment Period (all causalities) - Safety Population

| | tanezumab | tanezumab | |
|---|---|---|---|
| | 5 mg | 10 mg | celecoxib |
| | (N = 92) | (N = 93) | (N = 92) |
| Number (%) of Patients | n (%) | n (%) | n (%) |
| Nasopharyngitis | 14 (15.2) | 13 (14.0) | 6 (6.5) |
| Back pain | 4 (4.3) | 5 (5.4) | 4 (4.3) |
| Fall | 6 (6.5) | 4 (4.3) | 3 (3.3) |
| Contusion | 3 (3.3) | 4 (4.3) | 2 (2.2) |
| Arthralgia | 5 (5.4) | 3 (3.2) | 6 (6.5) |
| Intervertebral disc protrusion | 2 (2.2) | 3 (3.2) | 3 (3.3) |
| Headache | 2 (2.2) | 3 (3.2) | 2 (2.2) |
| Hypoaesthesia | 5 (5.4) | 2 (2.2) | 3 (3.3) |
| Pain in extremity | 3 (3.3) | 1 (1.1) | 3 (3.3) |
| Myalgia | 3 (3.3) | 1 (1.1) | 1 (1.1) |
| Pyrexia | 2 (2.2) | 1 (1.1) | 4 (4.3) |
| Diarrhoea | 1 (1.1) | 1 (1.1) | 3 (3.3) |
| Gastroenteritis | 1 (1.1) | 1 (1.1) | 3 (3.3) |
| Musculoskeletal pain | 3 (3.3) | 0 | 3 (3.3) |
| Pneumonia | 3 (3.3) | 0 | 1 (1.1) |
| Hypertension | 2 (2.2) | 0 | 3 (3.3) |

Adverse events are shown by descending frequency by tanezumab 10 mg group followed by tanezumab 5 mg group and celexocib group.

The frequency of adverse events of abnormal peripheral sensation during the treatment period was highest in the tanezumab 5 mg group (9.8% for tanezumab 5 mg, 4.3% for tanezumab 10 mg, and 4.3% for celecoxib). The frequency of adverse events of potential sympathetic dysfunction was highest in the celecoxib group (7.6% for celecoxib group, 4.3% for tanezumab 10 mg, and 3.3% for tanezumab 5 mg). No deaths were reported during the treatment period or the safety follow-up period. A total of 5 patients (2 patients in each tanezumab group and 1 patient in the celecoxib group) met criteria for adjudication of a joint safety event (Table 10). The incidence and observation-time adjusted rates of the composite joint safety endpoint (rapidly progressive osteoarthritis [RPOA], primary osteonecrosis, subchondral insufficiency fracture, pathological fracture) were highest in the tanezumab 10 mg group (2.2% and 21.0 events/1000 patient-years), followed by the tanezumab 5 mg group (1.1% and 8.9 events/1000 patient-years); there were no composite endpoints in the celecoxib group (0% and 0 event/1000 patient-years).

TABLE 10
Summary of patients with Adjudicated Joint Safety Outcomes during the study - Safety Population

| | tanezumab | tanezumab | |
|---|---|---|---|
| | 5 mg | 10 mg | celecoxib |
| | (N = 92) | (N = 93) | (N = 92) |
| Patients analyzed by the Adjudication Committee, n (%) | 2 (2.2%) | 2 (2.2%) | 1 (1.1%) |
| Composite Joint Safety Endpoint*, n (%) [95% CI] | 1 (1.1%) | 2 (2.2%) | 0 |
| | [0.0%, 5.9%] | [0.3%, 7.6%] | [0.0%, 3.9%] |
| RPOA, n (%) [95% CI] | 1 (1.1%) | 1 (1.1%) | 0 |
| | [0.0%, 5.9%] | [0.0%, 5.8%] | [0.0%, 3.9%] |
| RPOA type 1, n (%) [95% CI] | 1 (1.1%) | 0 | 0 |
| | [0.0%, 5.9%] | [0.0%, 3.9%] | [0.0%, 3.9%] |
| RPOA type 2, n (%) [95% CI] | 0 | 1 (1.1%) | 0 |
| | [0.0%, 3.9%] | [0.0%, 5.8%] | [0.0%, 3.9%] |
| Primary Osteonecrosis, n (%) [95% CI] | 0 | 0 | 0 |
| | [0.0%, 3.9%] | [0.0%, 3.9%] | [0.0%, 3.9%] |
| Pathological Fracture, n (%) [95% CI] | 0 | 0 | 0 |
| | [0.0%, 3.9%] | [0.0%, 3.9%] | [0.0%, 3.9%] |
| Subchondral Insufficiency Fracture, n (%) [95% CI] | 0 | 1 (1.1%) | 0 |
| | [0.0%, 3.9%] | [0.0%, 5.8%] | [0.0%, 3.9%] |
| Patients with Only Normal Progression of OA, n (%) | 0 | 0 | 0 |
| Patients with Other Joint Outcome, n (%) | 1 (1.1%) | 0 | 1 (1.1%) |

RPOA = rapidly progressive osteoarthritis,
OA = osteoarthritis,
CI = confidence interval
*The composite joint safety endpoint includes any subject with an adjudicated outcome of primary osteonecrosis, RPOA type 1 or type 2, subchondral insufficiency fracture, or pathological fracture. Includes adjudicated event up to the end of the safety follow-up period or 26 weeks after the end of the treatment period, whichever is later.

Of the 5 patients who had adjudicated joint safety endpoints across the three treatment groups, one patient in the tanezumab 10 mg group had an adjudicated event of RPOA type 2 which led to a total joint replacement. It was the only reported total joint replacement during the study observation period. The affected joint in this patient was a hip that was Kellgren Lawrence (KL) Grade 1 on the Screening x-ray. Another patient in the tanezumab 10 mg group had a joint safety event adjudicated to subchondral insufficiency fracture. The affected joint was a knee that was KL grade 2 on the Screening x-ray. One patient in the tanezumab 5 mg group had an adjudicated RPOA type 1 event. In this patient, both knee joints had adjudicated outcomes of RPOA type 1. Both knees had radiographic evidence of OA on the Screening x-ray (KL grade 1, right knee; KL grade 2, left knee). Two patients had a joint safety event adjudicated to the ‘Other’ adjudication category (i.e. no pre-specified composite joint safety endpoint). Among the two patients, one patient in the tanezumab 5 mg group had a pre-existing subchondral insufficiency fracture (subchondral insufficiency fracture present in Screening radiographs), and another patient in the celecoxib group had pre-existing arthroplasty.

### Efficacy

The efficacy endpoint of change from Baseline to Week 16 in the LBPI score, the RMDQ total score, and 50% responder in the LBPI score at Week 16 are shown in Table 11. Treatment with tanezumab 5 mg and 10 mg showed a numerically larger improvement for change from baseline to Week 16 for LBPI compared to the celecoxib group, and treatment with tanezumab 10 mg showed a numerically larger improvement for change from baseline to Week 16 for RMDQ compared to tanezumab 5 mg and celecoxib treatment.

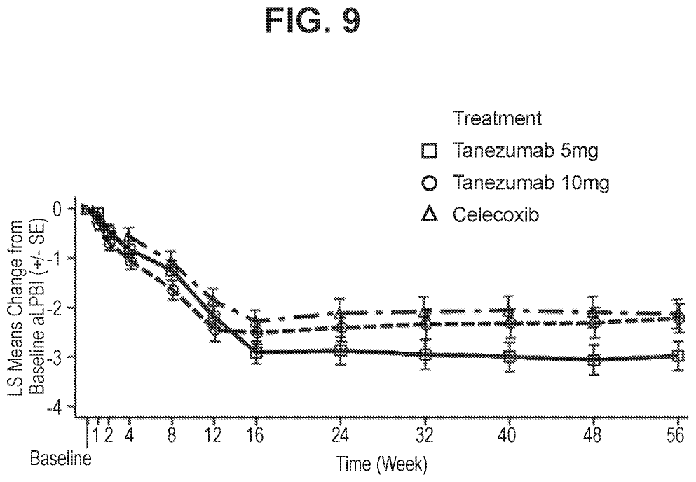

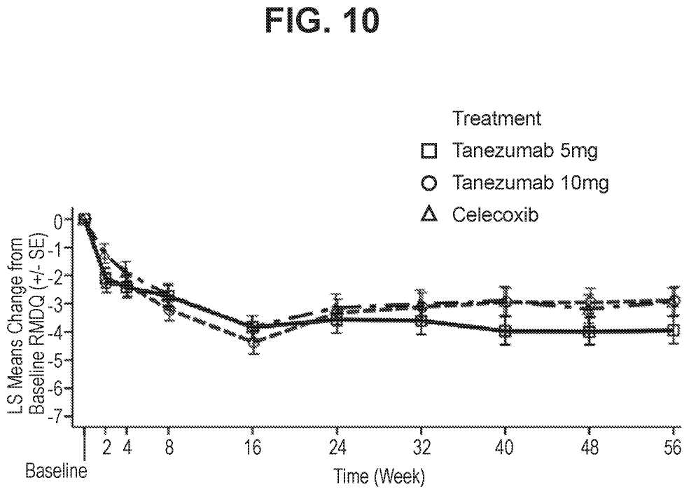

The change from Baseline for LBPI and RMDQ up to Week 56 are shown in FIG. 9 and FIG. 10. The treatment differences for change from baseline to Week 56 for LBPI and RMDQ were numerically larger in the tanezumab 5 mg group relative to both the tanezumab 10 mg and the celecoxib groups.

TABLE 11
Change from from Baseline for LBPI and RMDQ at week 16 (ITT)

| | | |
|---|---|---|
| tanezumab | tanezumab | |
| 5 mg | 10 mg | celecoxib |
| (N = 92) | (N = 93) | (N = 92) |

Change from Baseline to Week 16 in LBPI
(Multiple Imputation)

| | | | | | | |
|---|---|---|---|---|---|---|
| LS Mean (SE) | −2.91 | (0.23) | −2.51 | (0.23) | −2.28 | (0.23) |
| LS Mean Difference vs. | −0.63 | (−1.24, −0.03) | −0.23 | (−0.84, 0.38) | | |
| celecoxib (95% CI) | | | | | | |

Change from Baseline to Week 16 in RMDQ
(Multiple Imputation)

| | | | | | | |
|---|---|---|---|---|---|---|
| LS Mean (SE) | −3.85 | (0.41) | −4.38 | (0.42) | −3.84 | (0.42) |
| LS Mean Difference vs. | −0.01 | (−1.06, 1.05) | −0.53 | (−1.60, 0.53) | | |
| celecoxib (95% CI) | | | | | | |

Proportion of Patients with ≥50% Improvement
in LBPI at Week 16 (Mixed BOCF/LOCF)

| | | | | | | |
|---|---|---|---|---|---|---|
| n (%) | 47 | (51.1%) | 33 | (35.5%) | 30 | (32.6%) |
| Difference vs. | 18.5 | (4.2, 32.0) | 2.9 | (−10.7, 16.3) | | |
| celecoxib (95% CI) | | | | | | |

ITT = Intent-to-Treat,
LBPI = Low back pain intensity,
RMDQ = Roland Morris Disability Questionnaire,
SE = standard error,
CI = confidence interval
A change from baseline <0 is an improvement.

### Interpretation of Primary Results

The primary objective of the study was to evaluate the long-term safety of tanezumab 10 mg and 5 mg SC relative to celecoxib treatment over the course of 56-weeks of treatment. The safety profile of tanezumab treatment observed in this study was consistent with the earlier study (Example 1) conducted in patients with CLBP. The overall incidence of adverse events during the treatment period in both tanezumab groups (5 mg: 63.0%, 10 mg: 54.8%) was lower than in the celecoxib group (67.4%) during the 56-week treatment period. The incidence of serious adverse events during the 56-week treatment period was highest in the tanezumab 10 mg group (9.7%), followed by the tanezumab 5 mg group (4.3%) and the celecoxib group (2.2%). The highest incidence of discontinuations from treatment due to an adverse event during the treatment period occurred in the tanezumab 10 mg treatment group (5.4%) relative to the celecoxib (4.3%) and tanezumab 5 mg treatment groups (3.3%). No deaths were reported during Study 1063.

The incidence and observation-time adjusted rates of the composite joint safety endpoint were highest in the tanezumab 10 mg group (2.2% and 21.0 events/1000 patient-years), followed by the tanezumab 5 mg group (1.1% and 8.9 events/1000 patient-years) and the celecoxib group (0% and 0 event/1000 patient-years). Of the 3 patients included in the composite joint safety endpoint, one patient each in the tanezumab 10 mg group had an adjudication outcome of RPOA type 2 or subchondral insufficiency fracture, and one patient in the tanezumab 5 mg group had an adjudication outcome of RPOA type 1 during the study observation period.

Treatment with tanezumab 5 mg and 10 mg showed a numerically larger improvement for change from baseline to Week 16 for LBPI, and the treatment differences for change from baseline to Week 56 for LBPI and RMDQ were numerically larger in the tanezumab 5 mg group relative to both the tanezumab 10 mg and the celecoxib groups.

### Summary of Study 1063 (Example 2) and Study 1059 (Example 1)

Table 12 provides a summary of study design for CLBP Studies 1063 and 1059.

TABLE 12
Summary of study design and Key Entry criteria for CLBP studies 1063 and 1059

| | |
|---|---|
| Study 1063 (SC) | Study 1059 (SC) |

Study design

| Duration | Double-bline Treatment (56 weeks, 10 in-clinic visits) | |
|---|---|---|
| | Safety Follow-up (24 weeks, 2 in-clinic visits). | |
| Treatment arms/ | Tanezumab 5 mg, 10 mg, celecoxib | Tanezumab 5 mg, 10 mg, placebo, tramadol |
| randomiztion ratio | (1:1:1) | PR (2:2:2:2) |

Entry criteria

| | | |
|---|---|---|
| Requirements for | Be experiencing some benefit from | Documented history of previous inadequate |
| medication usage | their current stable dose regimen of | treatment response to at least 3 different |
| prior to Screening | oral therapy of NSAID (celecoxib 200 | categories of agents commonly used and |
| | mg/day [100 mg BID], loxoprofen 120 | generally considered effective for the |
| | to 180 mg/day or meloxicam 5 to 15 | treatment of CLBP: |
| | mg/day) treatment, be tolerating their | acetaminophen/low-dose NSAIDs |
| | NSAID regimen, be taking this | prescription NSAIDs |
| | medication regularly (defined as an | opioids (not tramadol) |
| | average of at least 5 days per week) | tapentadol, |
| | during the 30 day period prior to the | tricyclic antidepressants |
| | Screening visit and must have had | benzodiazepines or skeletal muscle |
| | some improvement in low back pain, | relaxants |
| | but still require additional pain relief at | lidocaine patch |
| | Screening | duloxetine or other serotonin- |
| | | norepinephrine reuptake inhibitors |

| | |
|---|---|
| LBPI at Baseline | ≥5 |
| PGA at Baseline | Fair, poor, or very poor |

CLBP = chronic low back pain;
NSAIDs = nonsteroidal anti-inflammatory drugs;
PGA = Patient's Global Assessment;
SC = subcutaneous,
SR = sustained release

The baseline characteristics across treatment groups are summarized for this study (1063) and the global CLBP study with tanezumab (1059) in Table 13. The range of mean scores across treatment groups for LBPI and RMDQ suggest the patients enrolled in Study 1063 (LBPI=6.72 to 6.82; RMDQ=7.75 to 8.27) had milder CLBP, particularly with respect to physical function, than the patients enrolled in Study 1059 (overall population LBPI=7.17 to 7.24; RMDQ=14.81 to 15.10, Japanese population LBPI=6.71 to 7.21; RMDQ=8.48 to 11.47). Study 1063 had approximately 10% fewer patients with degenerative joint disease/osteoarthritis (1063=14.8% vs. 1059=24.3%) and more patients with degenerative disc disease (1063=39.7% vs. 1059=30.1%). Study 1063 had a smaller proportion of patients with CLBP attributed to injury/muscular strain compared to Study 1059 (1063=2.5% vs. 1059=32.4%). Study 1063 included many CLBP patients whose etiology was reported as ‘Other’ (43.0%), and the cause of LBP in most of these patients (73.9%, 88/119) was not specified (i.e. unknown, non-specific LBP etc.). Based on assessments from the painDETECT screening tool to predict the likelihood of a neuropathic pain component being present in individual patients, 85.6% of the patients enrolled in Study 1063 had a predominantly non-neuropathic pain component (Study 1059: 68.4%) and approximately 6.1% likely had a neuropathic pain component (Study 1059: 12.6%).

The rate of completer of treatment phase (up to Week 56) in Study 1063 (5 mg: 67.4%, 10 mg: 46.2%) was higher than Study 1059 (5 mg: 34.4%, 10 mg: 39.1%).

TABLE 13
Summary of Key Baseline Characteristics
for CLBP Studies 1063 (SC) and 1059 (SC)

| | Study 1063 | Study 1059 |
|---|---|---|
| Baseline Characteristic (range of | (SC) | (SC) |
| mean scores across treatment groups) | (N = 277) | (N = 1825) |
| LBPI^{a} | 6.72-6.82 | 7.17-7.24 |
| RMDQ^{b} | 7.75-8.27 | 14.81-15.10 |
| PGA of CLBP^{c} | 3.13-3.24 | 3.47-3.53 |

Primary etiology assessment,^{d} n (%)

| Degenerative disc disease | 110 | (39.7) | 550 | (30.1) |
|---|---|---|---|---|
| Degenerative joint diease/OA | 41 | (14.8) | 443 | (24.3) |
| Injury/muscular strain | 7 | (2.5) | 591 | (32.4) |
| Injury/muscular strain: degenerative | 0 | | 1 | (0.1) |
| disc disease, herniated disc | | | | |
| Other | 119 | (43.0) | 240 | (13.2) |

painDETECT Category,^{e} n (%)

| | | | | |
|---|---|---|---|---|
| ≤12 (neuropathic component | 237 | (85.6) | 1249 | (68.4) |
| unlikely) | | | | |
| 13 to 18 (neuropathic component | 23 | (8.3) | 344 | (18.9) |
| uncertain) | | | | |
| ≥19 (neuropathic component likely) | 17 | (6.1) | 230 | (12.6) |

LBPI = Low Back Pain Intensity;
RMDQ = Roland Morris Disability Questionnaire;
SC = subcutaneous
^{a}Assessed with an 11-point numeric rating scale ranging from 0 (no pain) to 10 (worst possible pain)
^{b}CLBP-specific assessment of physical function with scores ranging from 0 to 24 (lower scores indicate better function).
^{c}Global evaluation that utilizes 5-point Likert scale with a score of 1 being best (very good) and a score of 5 being worst (very poor).
^{d}Principal Investigators' assessment of the primary etiology of the patients' CLBP based on patient report, history and physical examination, medical records or report from patient's physician, or imaging report.
^{e}Tool to screen for the prevalence of neuropathic pain components in CLBP patients, with scores ≤12 indicating that a neuropathic component is unlikely and scores ≥19 indicating a neuropathic component is likely. For scores of 13-19, the result is uncertain, i.e. a neuropathic pain component can be present. Range of scores −1 to 38.

### Safety

The adverse event profile including general safety and joint safety of tanezumab treatment observed in Japanese Study 1063 was generally consistent with the global CLBP Study 1059.

### General Safety

In Study 1063, the overall incidence of adverse events during the 56-week treatment period in the tanezumab treatment groups (5 mg: 63.0%, 10 mg: 54.8%) was lower than in the celecoxib treatment group (67.4%) (Study 1059: 5 mg; 58.3%, 10 mg; 63.7%). The incidence of serious adverse events during the 56-week treatment period was highest in the tanezumab 10 mg treatment group (9.7%), followed by the tanezumab 5 mg (4.3%) and celecoxib treatment groups (2.2%) (Ref. Study 1059: 5 mg; 2.2%, 10 mg; 4.6%). The highest incidence of discontinuations from treatment due to an adverse event during the 56-week treatment period occurred in the tanezumab 10 mg treatment group (5.4%) relative to the celecoxib (4.3%) and tanezumab 5 mg treatment groups (3.3%)(Study 1059:5 mg; 6.7%, 10 mg; 7.4%). No deaths were reported during Study 1063.

The most frequent adverse events (≥3% in any tanezumab treatment group in Study 1063) were nasopharyngitis, back pain, contusion, fall, arthralgia, headache, intervertebral disc protrusion, hypoaesthesia, myalgia, pain in extremity, musculoskeletal pain, pneumonia.

The incidence of adverse events of abnormal peripheral sensation was 9.8% in the tanezumab 5 mg treatment group, 4.3% in the tanezumab 10 mg treatment group and 4.3% in the celecoxib treatment group in Study 1063; the most common adverse events of abnormal peripheral sensation in Study 1063 was hypoaesthesia (5 mg: 5.4%, 10 mg: 2.2%[Study 1059 5 mg: 3.0%, 10 mg: 3.8%]) and carpal tunnel syndrome (5 mg: 1.1%, 10 mg: 2.2%[Study 1059 5 mg: 1.0%, 10 mg: 1.6%]); all abnormal peripheral sensation adverse events in the tanezumab treatment groups in Study 1063 were mild in severity.

The incidence of adverse events of potential sympathetic dysfunction was 3.3% in the tanezumab 5 mg group, 4.3% in the tanezumab 10 mg group and 7.6% in the celecoxib group in Study 1063(Study 1059 5 mg: 5.9%, 10 mg: 6.4%); all potential sympathetic dysfunction events in the tanezumab treatment groups in Study 1063 were mild in severity.

The results suggest the overall adverse event profile in Study 1063 was not notably different from global Study 1059.

### Joint Safety

Five patients (1.8% of total patients) met the criteria for adjudication of joint safety outcome during the 80-week observation period, and there was a higher number of tanezumab-treated patients (4/185=2.2% of patients) who met criteria for adjudication compared to celecoxib-treated patients (1/92=1.1%).

In the tanezumab 5 mg treatment group, one patient had an adjudicated event of RPOA Type 1(both right and left knees, baseline Kellgren Lawrence [KL] Grade 1 and Grade 2, respectively) and another patient had an adjudicated event of ‘Other’(an adjudication outcome other than the pre-specified categories)/pre-existing SIF (right knee, baseline KL Grade 1). In the tanezumab 10 mg treatment group, one patient had an adjudicated event of RPOA Type 2 (left hip, baseline KL Grade 1) and another patient had an adjudicated event of SIF (left knee, baseline KL Grade 2). In the celecoxib treatment group, one patient had an adjudicated event of Other/pre-existing arthroplasty (right knee, baseline KL Grade: NA).

Table 14 provides a summary of the composite joint safety endpoint and individual joint safety endpoints in the tanezumab treatment groups for CLBP Studies 1063 and 1059 and also provides data from a previous OA Study 1058 as a reference.

The incidence of the composite joint safety endpoint in the combined tanezumab treatment group in Study 1063 (1.6%) was similar to CLBP Study 1059 (1.8%), and it was lower than the previous OA Study 1058 (5.5%). Even though the patient number randomized to tanezumab treatment groups in Study 1063 (N=185) was approximately 18% of the size of Study 1059 (N=1008), the similar incidence of the composite joint safety endpoint suggests data from Study 1063 are generally consistent with Study 1059 and no additional joint safety signal was identified from Study 1063.

TABLE 14
Summary of Composite Joint Safety Endpoint and Individual Joint Safety Endpoints for
Tanezumab CLBP Studies 1063 and 1059 and OA Study 1058- Tanezumab Treatment Groups

| | CLBP Study | CLBP Study | | OA Study | |
|---|---|---|---|---|---|
| | 1063 | 1059 | | 1058 | |
| | tanezumab | tanezumab | | tanezumab | |
| | 5-10 mg | 5-10 mg | | 2.5-5 mg | |
| | N = 185 | N = 1008 | | N = 2000 | |
| Length of | 80 weeks/ | 80 weeks/ | | 80 weeks/ | |
| Study/Observation | 80 weeks/ | 80 weeks/ | | 80 weeks/ | |
| Period^{a}/study type | Japan local | Global | | Global | |
| Planned number of | 7 | 7 | | 7 | |
| injections of SC study | | | | | |
| medication | | | | | |
| Composite Joint Safety | 3 (1.6%) | 18 | (1.8%) | 109 | (5.5%) |
| Endpoint^{b}, n (%) | 5 mg = 1/92 (1.1%) | 5 mg = 5/506 (1.0%) | | 2.5 mg = 38/1002 (3.8%) | |
| | 10 mg = 2/93 (2.2%) | 10 mg = 13/502 (2.6%) | | 5 mg = 71/998 (7.1%) | |
| RPOA, n (%) | 2 (1.1%) | 14 | (1.4%) | 95 | (4.8%) |
| Type 1^{c} | 1 (0.5%) | 12 | (1.2%) | 78 | (3.9%) |
| Type 2^{d} | 1 (0.5%) | 2 | (0.2%) | 17 | (0.9%) |
| Primary Osteonecrosis, | 0 | 0 | | 1 | (0.1%) |
| n (%) | | | | | |
| Subchondral | 1 (0.5%) | 4 | (0.4%) | 13 | (0.7%) |
| Insufficiency Fracture, | | | | | |
| n (%) | | | | | |

^{a}Observation period for Studies 1058, 1059, and 1063 was the period from Baseline up to the end of the safety follow-up period or 26 weeks after end of the treatment period, whichever is later.
^{b}Rapidly progressive OA, primary osteonecrosis, subchondral insufficiency fracture, pathologic fracture
^{c}Accelerated joint space narrowing of ≥2 mm over approximately one year
^{d}Abnormal loss/destruction of bone that is not normally present in end-stage OA Composite joint safety endpoints observed in comparator treatment groups: No subject was observed in the celecoxib treatment group (N = 92) of Study 1063. No subject was observed in the placebo treatment group (N = 215) and 1 subject (RPOA Type 1) was observed in tramadol PR treatment group (N = 602) of Study 1059, and 15 sujects [RPOA Type 1: n = 11, RPOA Type 2: n − 1 and SIF: n = 4 (1 subject had both RPOA Type 1 and SIF)] were observed in NSAID treatment group (N = 996) of Study 1058.

### Efficacy

Treatment with tanezumab 5 mg and 10 mg provided a numerically larger improvement in change from baseline to week 16 for LBPI score compared to celecoxib treatment (Table 15). Study 1063 was generally consistent with Study 1059 in that the tanezumab treatment groups achieved numerically larger improvement than active comparator.

TABLE 15
Summary of Key Efficacy Endpoints for CLBP Studies
1063 and 1059 - ITT Population, Multiple Imputation

| Study 1063 | | | Study 1059 | | | |
|---|---|---|---|---|---|---|
| Tanezumab | Tanezumab | | | Tanezumab | Tanezumab | Tramadol |
| 5 mg | 10 mg | Celecoxib | Placebo | 5 mg | 10 mg | PR |
| (N = 92) | (N = 93) | (N = 92) | (N = 406) | (N = 407) | (N = 407) | (N = 605) |

Change from baseline to Week 16 in LBPI

| | | | | | | | |
|---|---|---|---|---|---|---|---|
| LS Mean | −2.91 | −2.51 | −2.28 | −2.68 | −2.98 | −3.08 | −2.81 |
| (SE) | (0.23) | (0.23) | (0.23) | (0.15) | (0.14) | (0.14) | (0.12) |

Change from baseline to Week 16 in RMDQ total score

| | | | | | | | |
|---|---|---|---|---|---|---|---|
| LS Mean | −3.85 | −4.38 | −3.84 | −4.95 | −6.27 | −6.69 | −5.21 |
| (SE) | (0.41) | (0.42) | (0.49) | (0.36) | (0.35) | (0.35) | (0.30) |

The improvement of LBPI and RMDQ total score from baseline to week 56 was numerically larger in the tanezumab 5 mg treatment group relative to both tanezumab 10 mg and the celecoxib treatment groups.

1. A method for treating chronic low back pain in a patient, the method comprising administering to the patient an anti-nerve growth factor (NGF) antibody at a dose of about 10 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

2. A method for treating chronic low back pain in a patient, the method comprising administering to the patient an anti-nerve growth factor (NGF) antibody at a dose of about 5 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

3. The method according to claim 1 or 2, wherein the anti-NGF antibody is tanezumab.

4. The method according to any one of claims 1 to 3, wherein the treatment effectively reduces low back pain intensity (LBPI).

5. The method according to any one of claims 1 to 4, wherein the treatment improves chronic low back pain as measured by: Roland Morris Disability Questionnaire (RMDQ); ≥30% improvement in LBPI at week 16; ≥50% improvement in LBPI at week 16; and/or reduction in LBPI from baseline to week 2 of treatment.

6. The method according to any one of claims 1 to 5, wherein the treatment effectively improves chronic low back pain at at least 24 weeks after start of treatment.

7. The method according to any one of claims 1 to 6, wherein the treatment effectively improves chronic low back pain at at least 56 weeks after start of treatment.

8. The method according to any one of the preceding claims, wherein the treatment effectively improves LBPI, RMDQ and/or PGA of low back pain compared to a baseline value prior to or at start of treatment.

9. The method according to any one of the preceding claims, wherein the treatment improves chronic low back pain compared to treatment with an opioid analgesic.

10. The method according to claim 9, wherein the treatment improves chronic low back pain compared to treatment with Tramadol.

11. The method according to any one of claims 1-8, wherein the treatment improves chronic low back pain compared to treatment with a NSAID.

12. The method according to claim 11, wherein the treatment improves chronic low back pain compared to treatment with celecoxib.

13. The method according to any one of the preceding claims, wherein the patient was previously treated with the analgesic therapy prior to administering the anti-NGF antibody.

14. The method according to any one of the preceding claims, wherein the prior therapy comprises at least three different categories of agents used for treatment of chronic low back pain.

15. The method according to any one of the preceding claims, wherein the patient has a history of inadequate pain relief from or intolerance to at least three different classes of analgesics.

16. The method according to claim 15, wherein the classes of analgesics comprise NSAIDs and opioids.

17. The method according to any one of the preceding claims, wherein the patient is not administered an NSAID during the treatment with the anti-NGF antibody.

18. The method according to any one of the preceding claims, wherein the patient is not administered a placebo during the treatment with the anti-NGF antibody.

19. The method according to any one of the preceding claims, wherein the patient is subjected to radiographic assessment of the knee, hip and/or shoulder prior to starting treatment with the anti-NGF antibody

20. The method according to claim 19, wherein if radiographic assessment identified rapidly progressive osteoarthritis of the joint, the patient is excluded from the treatment with the anti-NGF antibody.

21. The method of any one of the preceding claims, wherein the patient has moderate to severe chronic low back pain.

22. The method of any one of the preceding claims, wherein the patient has had chronic low back pain for at least three months.

23. The method according to any one of the preceding claims, wherein the patient, prior to administering the anti-NGF antibody, has a) low back pain with the primary location between the 12^{th} thoracic vertebra and the lower gluteal folds, classified as Category 1 (pain without radiation) or 2 (pain with proximal radiation [above the knee]) according to the classification of the Quebec Task Force in Spinal Disorders; b) a duration of chronic low back pain of at least three months; c) a Patient Global Assessment (PGA) measure of fair, poor, or very poor; and/or d) an average LBPI score of greater than 5.

24. The method according to any one of the preceding claims, wherein the patient, prior to administering the anti-NGF antibody, has radiographic evidence of knee osteoarthritis (Kellgren Lawrence Grade ≤2); and/or does not meet the American College of Rheumatology (ACR) clinical and radiographic criteria; and/or does not have pain associated with knee osteoarthritis.

25. The method according to any one of the preceding claims, wherein the patient prior to administering the anti-NGF antibody has no or possible radiographic evidence of hip osteoarthritis (Kellgren Lawrence Grade ≤1) and/or does not meet the American College of Rheumatology (ACR) clinical and radiographic criteria; and/or does not have pain associated with hip osteoarthritis.

26. The method according to any one of the preceding claims, wherein the patient prior to administering the anti-NGF antibody has no symptoms and radiographic evidence of osteoarthritis of the shoulder.

27. The method according to any one of the preceding claims, wherein the method further comprises conducting a radiographic assessment of the knee, hip and/or, shoulder at regular intervals.

28. The method according to any one of the preceding claims, wherein the anti-NGF antibody is administered for at least two or more doses at eight weekly intervals.

29. The method according to any one of the preceding claims, wherein the treatment with the anti-NGF antibody averts opioid addiction in the patient.

30. The method according to any one of the preceding claims, wherein the prior analgesic therapy comprises the administration of an opioid to the patient.

31. The method according to any one of the preceding claims, wherein the prior analgesic therapy comprises the administration of tramadol to the patient.

32. The method according to any one of the preceding claims, wherein the anti-NGF antibody comprises three CDRs from the variable heavy chain region having the sequence shown in SEQ ID NO: 1 and three CDRs from the variable light chain region having the sequence shown in SEQ ID NO: 2.

33. The method according to any one of the preceding claims, wherein the anti-NGF antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

34. The method according to any one of the preceding claims, wherein the anti-NGF antibody comprises a variable heavy chain region having the sequence shown in SEQ ID NO: 1 and a variable light chain region having the sequence shown in SEQ ID NO: 2.

35. The method according to any one of the preceding claims, wherein the anti-NGF antibody comprises a heavy chain having the sequence shown in SEQ ID NO: 9 and a light chain having the sequence shown in SEQ ID NO: 10, wherein the C-terminal lysine (K) of the heavy chain amino acid sequence of SEQ ID NO: 9 is optional.

36. The method according to any one of the preceding claims, wherein the anti-NGF antibody comprises a heavy chain having the sequence shown in SEQ ID NO: 11 and a light chain having the sequence shown in SEQ ID NO: 10.

37. The method according to any one of the preceding claims, wherein the anti-NGF antibody is administered to the patient for at least 80 weeks.

38. The method according to any one of the preceding claims, wherein the inadequate treatment comprises the administration of an NSAID, an opioid, and at least one of the following: a tapentadol, tricyclic antidepressants, benzodiazepine or other skeletal muscle relaxants, lidocaine, and duloxetine or other serotonin-norepinephrine reuptake inhibitors.

39. The method according to any one of the preceding claims, wherein the inadequate treatment comprises the administration of an NSAID, an opioid, and at least two of of the following: tapentadol, tricyclic antidepressants, benzodiazepine or other skeletal muscle relaxants, lidocaine, and duloxetine or other serotonin-norepinephrine reuptake inhibitors.

40. The method according to any one of the preceding claims, wherein the inadequate treatment comprises the administration of an NSAID, an opioid, and at least three of the following: tapentadol, tricyclic antidepressants, benzodiazepine or other skeletal muscle relaxants, lidocaine, and duloxetine or other serotonin-norepinephrine reuptake inhibitors.

41. An anti-NGF antibody for use in a method for treating chronic low back pain (CLBP) in a patient, the method comprising administering to the patient an anti-nerve growth factor (NGF) antibody at a dose of about 10 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

42. An anti-NGF antibody for use in a method for treating chronic low back pain (CLBP) in a patient, the method comprising administering to the patient an anti-nerve growth factor (NGF) antibody at a dose of about 5 mg every 8 weeks via subcutaneous injection; wherein the patient has a history of inadequate treatment response to prior therapy including analgesics and the treatment with the anti-NGF antibody effectively improves the chronic low back pain at least 16 weeks after start of treatment with the anti-NGF antibody.

43. An anti-NGF antibody for use of claim 41 or 42, wherein the method is as defined in any one of claims 3 to 40.
